(19) United States
(12) Patent Application Publication
(10) Pub. No.: US 2024/0279176 A1
(43) Pub. Date: Aug. 22, 2024

(54) SUBSTITUTED TRICYCLIC HETEROCYCLIC COMPOUNDS AS METALLO-BETA-LACTAMASE INHIBITORS

(71) Applicant: COUNCIL OF SCIENTIFIC & INDUSTRIAL RESEARCH, New Delhi (IN)

(72) Inventors: Vinod Dinkar CHAUDHARI, Chandigarh (IN); Pulkit DHIMAN, Chandigarh (IN); Hemraj Santuji NANDANWAR, Chandigarh (IN); Vikas PATHANIA, Chandigarh (IN); Krishan Gopal THAKUR, Chandigarh (IN); Satyajeet DAS, Chandigarh (IN)

(73) Assignee: COUNCIL OF SCIENTIFIC & INDUSTRIAL RESEARCH, New Delhi (IN)

(21) Appl. No.: 18/565,742

(22) PCT Filed: Jun. 2, 2022
(86) PCT No.: PCT/IN2022/050513

§ 371 Date: Nov. 30, 2023

(30) Foreign Application Priority Data
Jun. 3, 2021 (IN) 202111024755

(51) Int. Cl. C07D 209/60; A61K 31/403; A61K 31/407; A61K 31/4439; A61K 31/506; A61P 31/04; C07C 29/143; C07C 45/42; C07C 227/12; C07D 401/04; C07D 403/04

(52) U.S. Cl. CPC C07D 209/60; A61K 31/403; A61K 31/407; A61K 31/4439; A61K 31/506; A61P 31/04; C07C 29/143; C07C 45/42; C07C 227/12; C07D 401/04; C07D 403/04

(57) ABSTRACT

The invention relates to substituted tricyclic heterocyclic compounds of formula (I), pharmaceutically acceptable salts thereof and pharmaceutical compositions wherein, R1, R2, R3, R4, R5, R6, R7, R8, R9, R10, R11, X1, X2, X3, X4, X5 and ‘n’ defined herein. The present invention also relates to compositions which comprise a metallo-β-lactamase (MBL) inhibitor compound of the invention or a pharmaceutically acceptable salt thereof, and a pharmaceutically acceptable carrier, optionally in combination with a beta-lactam antibiotic and/or a beta-lactamase inhibitor. The invention further relates to methods for treating a bacterial infection comprising administering to a patient a therapeutically effective amount of a compound of the invention, in combination with a therapeutically effective amount of one or more β-lactam antibiotics and optionally in combination with one or more beta-lactamase inhibitor compounds. The compounds of the invention are useful in the methods described herein for reducing or overcoming antibiotic resistance.

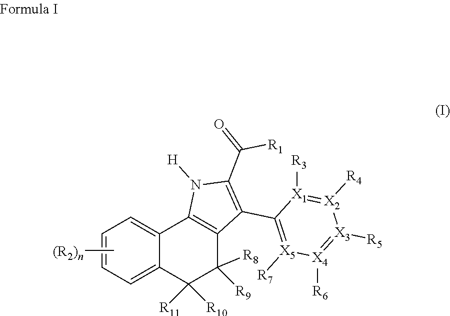

### FIELD OF THE INVENTION

Present invention relates to substituted tricyclic heterocyclic compound of formula (I), pharmaceutically acceptable salts thereof.

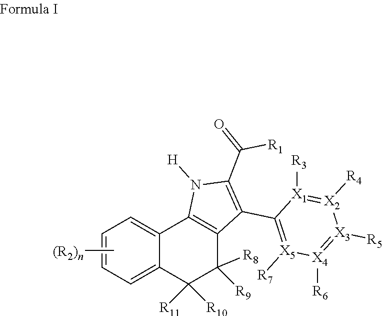

Particularly, present invention relates to substituted tricyclic heterocyclic compound of formula (I), pharmaceutically acceptable salts thereof useful as inhibitors of metallo-β-lactamase (MBL) enzyme and useful for reducing or removing antibiotic resistance bacteria. More particularly, present invention also relates to a process for the preparation of the compounds of formula I. Present invention further relates to a pharmaceutical composition containing the compounds of the formula (I), or pharmaceutically acceptable salts thereof.

### BACKGROUND OF THE INVENTION

Multidrug-resistant (MDR) organisms cause infections and their mortality rate is high as compared to infections caused by susceptible bacteria. Thousands of people are dying due to the infections caused by antibiotic-resistant organisms. Moreover, a recent report predicted that antibiotic resistance might be causing 300 million premature deaths by 2050. Resistance has emerged to all the known antibiotics resulting in increased infections that are untreatable and there is no alternative antibiotic. (Munita, J. et al, HS Public Access. 2016, 4(2), 1-37). Despite the numerous successes of the β-lactam antibiotics, bacteria have developed resistance to them and most troubling reason is β-lactamases, the bacterial enzyme. They react with antibiotics and hydrolyse the beta-lactam ring, which results in inactivation of beta Lactam antibiotics. Among Gram-negative bacteria, there are four classes of beta-lactamases, the serine beta-lactamases (SBLs) of the classes A, C and D, and the MBLs (class B). SBL enzymes uses active serine present in its catalytic site to hydrolyse β-lactam rings in a covalent mechanism whereas MBL enzymes requires Zn metal which helps in coordination and a hydroxide ion to hydrolyse the β-lactam ring. (Spencer, J., & Walsh, T. R; Angewandte Chemie—International Edition, 2006, 45(7), 1022-1026; E. Zhang et al., 2018). The zinc-dependent class B metallo-beta-lactamases are represented mainly by the NDM, VIM, and IMP types. IMP and VIM—producing K. pneumonia were first observed in 1990s in Japan and 2001 in Southern Europe, respectively. IMP—positive strains remain frequent in Japan and have also caused hospital outbreaks in China and Australia. However, dissemination of IMP-producing Enterobacteriaceae in the rest of the word appears to be somewhat limited. VIM producing enterobacteria can be frequently isolated in Mediterranean countries, reaching epidemic proportions in Greece. Isolation of VIM—producing strains remains low in Northern Europe and in the United States. In stark contrast, a characteristic of NDM—producing K. pneumonia isolates has been their rapid dissemination from their epicentre, the Indian subcontinent, to Western Europe, North America, Australia and Far East. Moreover, NDM genes have spread rapidly to various species other than K. pneumonia. To overcome the problem of increasing resistance, the need of the hour is to develop new compounds like inhibitors of β-lactamases which can enhance the efficacy of previously available antibiotics and are potent for both serine as well as MBLs. Meredith A. Hackel and his group discovered Vaborbactam, a potent inhibitor of KPCs and serine-lactamases. It has good potency for SBL but very less for MBLs (Hackel, M. A., et al; 2018, 1-10). Zheng et al, in PLOS One 2013, 8(5), e62955, discloses substituted 2,5-bistetrazolylmethyl-thiophenes and their use as β-lactamase inhibitors. (Zhang, Y. J., et al; Chemical and Pharmaceutical Bulletin, 2019, 67(2), 135-142; Zhang, E. et al, Bioorganic and Medicinal Chemistry Letters, 2018, 28(2), 214-221).

### OBJECTS OF THE INVENTION

Main object of the present invention is to provide substituted tricyclic heterocyclic compounds of formula (I), pharmaceutically acceptable salts thereof.

Another object of the present invention is to provide substituted tricyclic heterocyclic compounds of formula (I), pharmaceutically acceptable salts thereof useful as inhibitors of metallo-β-lactamase (MBL) enzyme and useful for reducing or removing antibiotic resistance bacteria.

Yet another object of the present invention is to provide a pharmaceutical composition containing the compounds of the formula (I), or pharmaceutically acceptable salts thereof

Yet another object of the present invention is to provide a process for the preparation of the compound of formula I.

### SUMMARY OF THE INVENTION

Accordingly, present invention provides a compound of formula I, a stereoisomer thereof, or a pharmaceutically acceptable salt thereof,

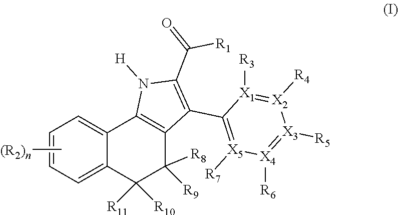

wherein,
R₁ is selected from the group consisting of
   (i) —OH;
   (ii) (ii) —NRₐR_{b}, wherein Rₐ and R_{b} are independently selected at each occurrence from hydrogen, unsubstituted or substituted alkyl, and unsubstituted or substituted cycloalkyl; methoxy; or Rₐ and R_{b} together with the carbon atom to which they are attached, may form a substituted or unsubstituted 3 to 7 membered saturated carbocyclic ring;
   (iii) —OR_{c}, wherein R_{c} is selected from C₁₋₆ alkyl, C₃₋₈ cycloalkyl, C₃₋₈ cycloalkyl, C₁₋₂ alkyl, aryl, aryl-C₁₋₂ alkyl, heteroaryl, heteroaryl-C₁₋₂ alkyl, heterocyclyl or heterocyclyl-C₁₋₂ alkyl, each of which is optionally substituted by one or more substituent groups;
   (iv) —R_{d}, wherein R_{d} is independently selected at each occurrence from hydrogen, substituted or unsubstituted alkyl, and substituted or unsubstituted cycloalkyl;
R₂, which may be same or different at each occurrence, is independently selected from the group consisting of halogen, cyano, nitro, substituted or unsubstituted alkyl, substituted or unsubstituted haloalkyl, substituted or unsubstituted cycloalkyl, —C(O)ORₑ, —OC(O)ORₑ, —O(CRₐR_{b})r-C(O)ORₑ, —(CRₐR_{b})ᵣ—C(O)ORₑ, —C(O)Rₕ, NR_{f}R_{g}, —C(O)NR_{f}R_{g}, —NR_{f}C(O)R_{b}, —NR_{f}S(O)₂R_{g}, —S(O)₀₋₂Rₑ, and —S(O)₂NR_{f}R_{g},
Rₐ and R_{b}, which may be same or different at each occurrence, are independently selected from the group consisting of hydrogen, halogen, hydroxy, cyano, substituted or unsubstituted haloalkyl, substituted or unsubstituted alkyl and substituted or unsubstituted cycloalkyl; or Rₐ and R_{b} together with the carbon atom to which they are attached, may form a unsubstituted or substituted 3 to 7 membered saturated carbocyclic ring; Rₑ, which may be same or different at each occurrence, is independently selected from the group consisting of hydrogen, substituted or unsubstituted aryl, substituted or unsubstituted haloalkyl, and substituted or unsubstituted alkyl;
R_{f} and R_{g}, which may be same or different at each occurrence, are independently selected from the group consisting of hydrogen, substituted or unsubstituted alkyl, —(CRaRb)ᵣ-C(O)ORₑ, substituted or unsubstituted cycloalkyl, substituted or unsubstituted cycloalkylalkyl, substituted or unsubstituted aryl, substituted or unsubstituted arylalkyl, substituted or unsubstituted heteroaryl, substituted or unsubstituted heteroarylalkyl, substituted or unsubstituted heterocyclyl, and substituted or unsubstituted heterocyclylalkyl; or R_{f} and R_{g} together with the nitrogen atom to which they are attached, may form a substituted or unsubstituted, saturated or unsaturated 3 to 10 membered cyclic ring, wherein the unsaturated cyclic ring may have one or two double bonds; at each occurrence, Rₕ is substituted or unsubstituted alkyl or substituted or unsubstituted aryl;
‘n’ is an integer ranging from 0 to 5, both inclusive;
‘r’ is an integer ranging from 1 to 3, both inclusive;
X₁, X₂, X₃, X₃ or X₅ are selected from C or N, with the proviso that only one or two of X₁, X₂, X₃, X₃ or X₅ can be N;
R₃ is selected from hydrogen, halo, cyano, nitro, hydroxy or a group-A¹-B¹-C¹
wherein, A¹ is absent or a linker group of the formula —[CR^{1A}R^{1B}]ₚ— in which p is an integer selected from 1 or 2, 3 or 4, and R^{1A} and R^{1B} are each independently selected from hydrogen or C₁₋₂ alkyl;
B¹ is absent or —O—, —C(O)—, —C(O)O—, —OC(O)—, —CH(OR^{1C})—, —N(R^{1C})—, N(R^{1D})—C(O), —N(R^{1D})—C(O)O—, —C(O)—N(R^{1C})—, —N(R^{1D})C(O)N(R^{1C})—, —S—, —SO—, —SO₂—, —S(O)₂N(R^{1C})—, or —N(R^{1D})SO₂— wherein R^{1C} and R^{1D} are each independently selected from hydrogen or methyl; and
C¹ is hydrogen, C₁₋₆ alkyl, C₂₋₆ alkenyl, C₂₋₆ alkynyl, aryl, C₃₋₆ cycloalkyl, C₃₋₆ cycloalkenyl, heterocyclyl, or heteroaryl;
and wherein C¹ is optionally further substituted by one or more substituent groups independently selected from oxo, halo, cyano, nitro, hydroxy, carboxy, NR^{1E}R^{1F}, C₁₋₄ alkoxy, C₁₋₄ alkyl, C₃₋₈ cycloalkyl, C₃₋₈ cycloalkyl-C₁₋₃ alkyl, C₁₋₄ alkanoyl, C₁₋₄ alkylsulphonyl, aryl, aryloxy, aryl-C₁₋₂ alkyl, heterocyclyl, heterocyclyloxy, heterocyclyl-C₁₋₂ alkyl, heteroaryl, heteroaryloxy, heteroaryl-C₁₋₂ alkyl, C(O)NR^{1E}R^{1F}, NR^{1E}C(O)R^{1F}, NR^{1E}S(O)₂R^{1F} and S(O)₂NR^{1E}R^{1F}; wherein R^{1E} and R^{1F} are each independently selected from hydrogen, C₁₋₄ alkyl or C₃₋₆ cycloalkyl or C₃₋₆ cycloalkyl C₁₋₂ alkyl; or R^{1E} and R^{1F} can be linked such that, together with the nitrogen atom to which they are attached, they form a 4-7 membered heterocyclic ring;
and wherein any alkyl, aryl, heterocyclyl or heteroaryl group present in a substituent group on C¹ is optionally further substituted by halo, cyano, nitro, hydroxy, caboxy, NR^{1G}R^{1H}, C₁₋₂ alkoxy, or C₁₋₂ alkyl; wherein R^{1G} and R^{1H} are selected from hydrogen or C₁₋₂ alkyl;
or R^{1C} and C¹ can be linked such that, together with the nitrogen atom to which they are attached, they form a 4-7 membered heterocyclic ring, which is optionally substituted by oxo, halo, cyano, nitro, hydroxy, carboxy, NR^{1E}R^{1F}, C₁₋₄ alkoxy, C₁₋₄ alkyl, C₃₋₈ cycloalkyl, C₃₋₈ cycloalkyl-C₁₋₃ alkyl, C₁₋₄ alkanoyl, C₁₋₄ alkylsulphonyl, or C(O)NR^{1E}R^{1F}, NR^{1E}C(O)R^{1F}, NR^{1E}S(O)₂R^{1F} and S(O)₂NR^{1E}R^{1F};
R₄ is selected from hydrogen, cyano, halo, nitro, hydroxy or a group
-A²-B²-C²
wherein, A² is absent or a linker group of the formula —[CR^{2A}R^{2B}]_{q}— in which q is an integer selected from 1 or 2, 3 or 4, and R^{2A} and R^{2B} are each independently selected from hydrogen or C₁₋₂ alkyl;
B² is absent or —O—, —C(O)—, —C(O)O—, —OC(O)—, —CH(OR^{2C})—, —N(R^{2C})—, N(R^{2D})—C(O)—, —N(R^{2D})—C(O)O—, —C(O)—N(R^{2C})—, —N(R^{2D})C(O)N(R^{2C})—, —S—, —SO—, —SO₂—, —S(O)₂N(R^{2C})—, or —N(R^{2D})SO₂— wherein R^{2C} and R^{2D} are each independently selected from hydrogen or methyl; and
C² is hydrogen, C₁₋₆ alkyl, C₂₋₆ alkenyl, C₂₋₆ alkynyl, aryl, C₃₋₄ cycloalkyl, C₃₋₆ cycloalkenyl, heterocyclyl; or heteroaryl
and wherein C² is optionally further substituted by one or more substituent groups independently selected from oxo, halo, cyano, nitro, hydroxy, carboxy, NR^{2E}R^{2F}, C₁₋₄ alkoxy, C₃₋₈ cycloalkyl, C₁₋₄ alkyl, C₃₋₈ cycloalkyl-C₁₋₃ alkyl, C₁₋₄ alkylsulphonyl, C₁₋₄ alkanoyl, aryl, aryloxy, aryl-C₁₋₂ alkyl, heterocyclyl, heterocyclyloxy, heterocyclyl-C₁₋₂ alkyl, heteroaryl, heteroaryloxy, heteroaryl-C₁₋₂ alkyl, C(O)NR^{2E}R^{2F}, NR^{2E}C(O)R^{2F}, NR^{2E}S(O)₂R^{2F} and S(O)₂NR^{2E}R^{2F}; wherein R^{2E} and R^{2F} are each independently selected from hydrogen, C₁₋₄ alkyl or C₃₋₆ cycloalkyl or C₃₋₄ cycloalkyl C₁₋₂ alkyl; or R^{2E} and R^{2F} can be linked such that, together with the nitrogen atom to which they are attached, they form a 4-7 membered heterocyclic ring;
and wherein any alkyl, aryl, heterocyclyl or heteroaryl group present in a substituent group on C² is optionally further substituted by halo, cyano, nitro, hydroxy, caboxy, NR^{2G}R^{2H}, C₁₋₂ alkoxy, or C₁₋₂ alkyl; wherein R^{2G} and R^{2H} are selected from hydrogen or C₁₋₂ alkyl;
or R^{2C} and C² can be linked such that, together with the nitrogen atom to which they are attached, they form a 4-7 membered heterocyclic ring, which is optionally substituted by oxo, halo, cyano, nitro, hydroxy, carboxy, NR^{2E}R^{2F}, C₁₋₄alkoxy, C₁₋₄ alkyl, C₃₋₈cycloalkyl, C₃₋₈cycloalkyl-C₁₋₃alkyl, C₁₋₄alkanoyl, C₁₋₄alkylsulphonyl, or C(O)NR^{2E}R^{2F}, NR^{2E}C(O)R^{2F}, NR^{2E}S(O)₂R^{2F} and S(O)₂NR^{2E}R^{2F};
R₅ is selected from hydrogen, halo, cyano, hydroxy, nitro, or a group
-A³-B³-C³
Wherein, A³ is absent or a linker group of the formula —[CR^{3A}R^{3B}]ᵣ in which r is an integer selected from 1 or 2, 3 or 4, and R^{3A} and R^{3B} are each independently selected from hydrogen or C₁₋₂ alkyl;
B³ is absent or —O—, —C(O)—, —C(O)O—, —OC(O)—, —CH(OR^{3C})—, —N(R^{3D})—, N(R^{3D})—C(O)—, —N(R^{3D})—C(O)O—, —C(O)—N(R^{3C})—, —N(R^{3D})C(O)N(R^{3C})—, —S—, —SO—, —SO₂—, —S(O)₂N(R^{3C})—, or —N(R^{3D})SO₂— wherein R^{3C} and R^{3D} are each independently selected from hydrogen or methyl; and
C³ is hydrogen, C₁₋₆ alkyl, C₂₋₆ alkenyl, C₂₋₆ alkynyl, aryl, C₃₋₄ cycloalkyl, C₃₋₄ cycloalkenyl, heteroaryl or heterocyclyl;
and wherein C³ is optionally further substituted by one or more substituent groups independently selected from oxo, halo, cyano, nitro, hydroxy, carboxy, NR^{3E}R^{3F}, C₁₋₄ alkoxy, C₁₋₄ alkyl, C₃₋₈ cycloalkyl, C₃₋₈ cycloalkyl-C₁₋₃ alkyl, C₁₋₄ alkanoyl, C₁₋₄ alkylsulphonyl, aryl, aryloxy, aryl-C₁₋₂ alkyl, heterocyclyl, heterocyclyloxy, heterocyclyl-C₁₋₂ alkyl, heteroaryl, heteroaryloxy, heteroaryl-C₁₋₂ alkyl, C(O)NR^{3E}R^{3F}, NR^{3E}C(O)R^{3F}, NR^{3E}S(O)₂R^{3F} and S(O)₂NR^{3E}R^{3F}; wherein R^{3E} and R^{3F} are each independently selected from hydrogen, C₁₋₄ alkyl or C₃₋₆ cycloalkyl or C₃₋₄ cycloalkyl C₁₋₂ alkyl; or R^{3E} and R^{3F} can be linked such that, together with the nitrogen atom to which they are attached, they form a 4-7 membered heterocyclic ring;
and wherein any alkyl, aryl, heterocyclyl or heteroaryl group present in a substituent group on C³ is optionally further substituted by halo, nitro, cyano, hydroxy, caboxy, NR^{3G}R^{3H}, C₁₋₂ alkoxy, or C₁₋₂ alkyl; wherein R^{3G} and R^{3H} are selected from hydrogen or C₁₋₂ alkyl;
or R^{3C} and C³ can be linked such that, together with the nitrogen atom to which they are attached, they form a 4-7 membered heterocyclic ring, which is optionally substituted by oxo, halo, cyano, hydroxy, nitro, carboxy, NR^{3E}R^{3F}, C₁₋₄ alkoxy, C₁₋₄ alkyl, C₃₋₈ cycloalkyl, C₃₋₈ cycloalkyl-C₁₋₃ alkyl, C₁₋₄ alkanoyl, C₁₋₄ alkylsulphonyl, or C(O)NR^{3E}R^{3F}, NR^{3E}C(O)R^{3F}, NR^{3E}S(O)₂R^{3F} and S(O)₂NR^{3E}R^{3F};
R₆ is selected from hydrogen, halo, cyano, nitro, hydroxy or a group
-A⁴-B⁴-C⁴
wherein, A⁴ is absent or a linker group of the formula —[CR^{4A}R^{4B}]ₛ— in which is an integer selected from 1 or 2, 3 or 4, and R^{4A} and R^{4B} are each independently selected from hydrogen or C₁₋₂ alkyl;
B⁴ is absent or —O—, —C(O)—, —C(O)O—, —OC(O)—, —CH(OR^{4C})—, —N(R^{4C})—, N(R^{4D})—C(O)—, —N(R^{4D})—C(O)O—, —C(O)—N(R^{4C})—, —N(R^{4D})C(O)N(R^{4C})—, —S—, —SO—, —SO₂—, —S(O)₂N(R⁴)—, or —N(R^{D})SO₂— wherein R^{4C} and R^{4D} are each independently selected from hydrogen or methyl; and
C⁴ is hydrogen, C₁₋₄ alkyl, C₂₋₆ alkenyl, C₂₋₆ alkynyl, aryl, C₃₋₆ cycloalkyl, C₃₋₄ cycloalkenyl, heterocyclyl, or heteroaryl,
and wherein C⁴ is optionally further substituted by one or more substituent groups independently selected from oxo, halo, cyano, nitro, hydroxy, carboxy, NR^{4E}R^{4F}, C₁₋₄ alkoxy, C₁₋₄ alkyl, C₃₋₈ cycloalkyl, C₃₋₈ cycloalkyl-C₁₋₃ alkyl, C₁₋₄ alkanoyl, C₁₋₄ alkylsulphonyl, aryl, aryloxy, aryl-C₁₋₂ alkyl, heterocyclyl, heterocyclyloxy, heterocyclyl-C₁₋₂ alkyl, heteroaryl, heteroaryloxy, heteroaryl-C₁₋₂ alkyl, C(O)NR^{4E}R^{4F}, NR^{4E}C(O)R^{4F}, NR^{4E}S(O)₂R^{4F} and S(O)₂NR^{4E}R^{4F}; wherein R^{4E} and R^{4F} are each independently selected from hydrogen, C₁₋₄ alkyl or C₃₋₆ cycloalkyl or C₃₋₆ cycloalkyl C₁₋₂ alkyl; or R^{4E} and R^{4F} can be linked such that, together with the nitrogen atom to which they are attached, they form a 4-7 membered heterocyclic ring;
and wherein any alkyl, aryl, heterocyclyl or heteroaryl group present in a substituent group on C⁴ is optionally further substituted by halo, cyano, nitro, hydroxy, caboxy, NR^{4G}R^{4H}, C₁₋₂ alkoxy, or C₁₋₂ alkyl; wherein R^{4G} and R^{4H} are selected from hydrogen or C₁₋₂ alkyl;
or R^{4C} and C⁴ can be linked such that, together with the nitrogen atom to which they are attached, they form a 4-7 membered heterocyclic ring, which is optionally substituted by oxo, halo, cyano, nitro, hydroxy, carboxy, NR^{4E}R^{4F}, C₁₋₄ alkoxy, C₁₋₄ alkyl, C₃₋₈ cycloalkyl, C₃₋₈ cycloalkyl-C₁₋₃ alkyl, C₁₋₄ alkanoyl, C₁₋₄ alkylsulphonyl, or C(O)NR^{4E}R^{4F}, NR^{4E}C(O)R^{4F}, NR^{4E}S(O)₂R^{4F} and S(O)₂NR^{4E}R^{4F};
R₇ is selected from hydrogen, halo, nitro, cyano, hydroxy or a group
-A⁵-B⁵-C⁵
Wherein, A⁵ is absent or a linker group of the formula —[CR^{5A}R^{5B}]ₜ— in which ‘t’ is an integer selected from 1 or 2, 3 or 4, and R^{5A} and R^{5B} are each independently selected from hydrogen or C₁₋₂ alkyl;
B⁵ is absent or —O—, —C(O)—, —C(O)O—, —OC(O)—, —CH(OR^{5C})—, —N(R^{5C})—, N(R^{5D})—C(O)—, —N(R^{5D})—C(O)O—, —C(O)—N(R^{5C})—, —N(R^{5D})C(O)N(R^{5C})—, —S—, —SO—, —SO₂—, —S(O)₂N(R^{5D})—, or —N(R^{5C})SO₂— wherein R^{5C} and R^{5D} are each independently selected from hydrogen or methyl; and
C⁵ is hydrogen, C₁₋₆ alkyl, C₂₋₆ alkenyl, C₂₋₆ alkynyl, aryl, C₃₋₄ cycloalkyl, C₃₋₄ cycloalkenyl, heteroaryl or heterocyclyl;
and wherein C⁵ is optionally further substituted by one or more substituent groups independently selected from oxo, cyano, halo, nitro, carboxy, hydroxy, NR^{5E}R^{5F}, C₁₋₄ alkoxy, C₁₋₄ alkyl, C₃₋₈ cycloalkyl, C₃₋₈ cycloalkyl-C₁₋₃ alkyl, C₁₋₄ alkanoyl, C₁₋₄ alkylsulphonyl, aryl, aryloxy, aryl-C₁₋₂ alkyl, heterocyclyl, heterocyclyloxy, heterocyclyl-C₁₋₂ alkyl, heteroaryl, heteroaryloxy, heteroaryl C₁₋₂ alkyl, C(O)NR^{5E}R^{5F}, NR^{5E}C(O)R^{5F}, NR^{5E}S(O)₂R^{5F} and S(O)₂NR^{5E}R^{5F}; wherein R^{5E} and R^{5F} are each independently selected from hydrogen, C₁₋₄ alkyl or C₃₋₄ cycloalkyl or C₃₋₆ cycloalkyl C₁₋₂ alkyl; or R^{5E} and R^{5F} can be linked such that, together with the nitrogen atom to which they are attached, they form a 4-7 membered heterocyclic ring; and wherein any alkyl, aryl, heterocyclyl or heteroaryl group present in a substituent group on C⁵ is optionally further substituted by halo, cyano, nitro, hydroxy, caboxy, NR^{5G}R^{5H}, C₁₋₂ alkoxy, or C₁₋₂ alkyl; wherein R^{5G} and R^{5H} are selected from hydrogen or C₁₋₂ alkyl;
or R^{5C} and C⁵ can be linked such that, together with the nitrogen atom to which they are attached, they form a 4-7 membered heterocyclic ring, which is optionally substituted by oxo, halo, cyano, nitro, hydroxy, carboxy, NR^{5E}R^{5F}, C₁₋₄ alkoxy, C₁₋₄ alkyl, C₃₋₈ cycloalkyl, C₃₋₈ cycloalkyl-C₁₋₃ alkyl, C₁₋₄ alkanoyl, C₁₋₄ alkylsulphonyl, or C(O)NR^{5E}R^{5F}, NR^{5E}EC(O)R^{5F}, NR^{5E}S(O)₂R^{5F} and S(O)₂NR^{5E}R^{5F};
R₈ and R₉, are selected from group, which may be same or different at each occurrence, are independently selected from hydrogen, hydroxy, halogen, cyano, substituted or unsubstituted haloalkyl, substituted or unsubstituted alkyl and substituted or unsubstituted cycloalkyl; or R₈ and R₉ together with the carbon atom to which they are attached, may form a substituted or unsubstituted 3 to 7 membered saturated carbocyclic ring; R₁₀ and R₁₁, which may be same or different at each occurrence, are independently selected from hydrogen, cyano, halogen, hydroxy, substituted or unsubstituted haloalkyl, substituted or unsubstituted alkyl and substituted or unsubstituted cycloalkyl; or R₁₀ and R₁₁ together with the carbon atom to which they are attached, may form a substituted or unsubstituted 3 to 7 membered saturated carbocyclic ring.

In an embodiment of the present invention, compound of formula I is selected from the group consisting of:
1. Methyl 3-(3,5-dichlorophenyl)-4,5-dihydro-1H-benzo[g]indole-2-carboxylate;
2. Methyl 3-(3,5-dichlorophenyl)-4,5-dihydro-1H-benzo[g]indole-2-carboxylic acid;
3. Methyl 3-(5-fluoro-2-methoxyphenyl)-4,5-dihydro-1H-benzo[g]indole-2-carboxylate;
4. Methyl 3-(2-methoxyphenyl)-4,5-dihydro-1H-benzo[g]indole-2-carboxylate;
5. Methyl 3-(4-methoxyphenyl)-4,5-dihydro-1H-benzo[g]indole-2-carboxylate;
6. Methyl 3-(3,4-difluorophenyl)-4,5-dihydro-1H-benzo[g]indole-2-carboxylate;
7. Methyl 3-(5-chloro-2-methoxyphenyl)-4,5-dihydro-1H-benzo[g]indole-2-carboxylate;
8. Methyl 3-(4-(tert-butyl)phenyl)-4,5-dihydro-1H-benzo[g]indole-2-carboxylate;
9. Methyl 3-(4-fluorophenyl)-4,5-dihydro-1H-benzo[g]indole-2-carboxylate;
10. Methyl 3-phenyl-4,5-dihydro-1H-benzo[g]indole-2-carboxylate;
11. Methyl 3-(2-fluorophenyl)-4,5-dihydro-1H-benzo[g]indole-2-carboxylate;
12. Methyl 3-(3-fluorophenyl)-4,5-dihydro-1H-benzo[g]indole-2-carboxylate;
13. Methyl 3-(4-chlorophenyl)-4,5-dihydro-1H-benzo[g]indole-2-carboxylate;
14. Methyl 3-(2-chlorophenyl)-4,5-dihydro-1H-benzo[g]indole-2-carboxylate;
15. Methyl 3-(3-chlorophenyl)-4,5-dihydro-1H-benzo[g]indole-2-carboxylate;
16. Methyl 3-(2,4-dichlorophenyl)-4,5-dihydro-1H-benzo[g]indole-2-carboxylate;
17. Methyl 3-(2,3-difluorophenyl)-4,5-dihydro-1H-benzo[g]indole-2-carboxylate;
18. Methyl 3-(3,5-dimethylphenyl)-4,5-dihydro-1H-benzo[g]indole-2-carboxylate;
19. Methyl 3-(2-fluoro-4-methylphenyl)-4,5-dihydro-1H-benzo[g]indole-2-carboxylate;
20. Methyl 3-(2,3-dichlorophenyl)-4,5-dihydro-1H-benzo[g]indole-2-carboxylate;
21. Methyl 3-(pyridin-4-yl)-4,5-dihydro-1H-benzo[g]indole-2-carboxylate;
22. Methyl 3-(3,5-difluorophenyl)-4,5-dihydro-1H-benzo[g]indole-2-carboxylate;
23. Methyl 3-(pyrimidin-5-yl)-4,5-dihydro-1H-benzo[g]indole-2-carboxylate;
24. Methyl 3-(5-fluoropyridin-3-yl)-4,5-dihydro-1H-benzo[g]indole-2-carboxylate;
25. Methyl 3-(5-chloropyridin-3-yl)-4,5-dihydro-1H-benzo[g]indole-2-carboxylate;
26. Methyl 3-(4-fluoro-3-methoxyphenyl)-4,5-dihydro-1H-benzo[g]indole-2-carboxylate;
27. Methyl 3-(3-chloro-5-methylphenyl)-4,5-dihydro-1H-benzo[g]indole-2-carboxylate;
28. Methyl 3-(3-chloro-5-fluorophenyl)-4,5-dihydro-1H-benzo[g]indole-2-carboxylate;
29. Methyl 3-(3-chloro-5-methoxyphenyl)-4,5-dihydro-1H-benzo[g]indole-2-carboxylate;
30. Methyl 3-(3,4,5-trichlorophenyl)-4,5-dihydro-1H-benzo[g]indole-2-carboxylate;
31. Methyl 3-(3,5-dimethoxyphenyl)-4,5-dihydro-1H-benzo[g]indole-2-carboxylate;
32. Methyl 3-(3-fluoro-5-methoxyphenyl)-4,5-dihydro-1H-benzo[g]indole-2-carboxylate;
33. Methyl 3-(2,3-dichlorophenyl)-4,5-dihydro-1H-benzo[g]indole-2-carboxylate;
34. 3-(5-Fluoro-2-methoxyphenyl)-4,5-dihydro-1H-benzo[g]indole-2-carboxylic acid;
35. 3-(2-methoxyphenyl)-4,5-dihydro-1H-benzo[g]indole-2-carboxylic acid;
36. 3-(4-Methoxyphenyl)-4,5-dihydro-1H-benzo[g]indole-2-carboxylic acid;
37. 3-(3,4-Difluorophenyl)-4,5-dihydro-1H-benzo[g]indole-2-carboxylic acid;
38. 3-(5-Chloro-2-methoxyphenyl)-4,5-dihydro-1H-benzo[g]indole-2-carboxylic acid;
39. 3-(4-(tert-Butyl)phenyl)-4,5-dihydro-1H-benzo[g]indole-2-carboxylic acid;
40. 3-(4-Fluorophenyl)-4,5-dihydro-1H-benzo[g]indole-2-carboxylic acid;
41. 3-(2-Fluorophenyl)-4,5-dihydro-1H-benzo[g]indole-2-carboxylic acid;
42. 3-(3-Fluorophenyl)-4,5-dihydro-1H-benzo[g]indole-2-carboxylic acid;
43. 3-(4-chlorophenyl)-4,5-dihydro-1H-benzo[g]indole-2-carboxylic acid;
44. 3-(2-chlorophenyl)-4,5-dihydro-1H-benzo[g]indole-2-carboxylic acid;
45. 3-(3-chlorophenyl)-4,5-dihydro-1H-benzo[g]indole-2-carboxylic acid;
46. 3-(2,4-dichlorophenyl)-4,5-dihydro-1H-benzo[g]indole-2-carboxylic acid;
47. 3-(2,3-difluorophenyl)-4,5-dihydro-1H-benzo[g]indole-2-carboxylic acid;
48. 3-(3,5-dimethylphenyl)-4,5-dihydro-1H-benzo[g]indole-2-carboxylic acid;
49. 3-(2-fluoro-4-methylphenyl)-4,5-dihydro-1H-benzo[g]indole-2-carboxylic acid;
50. 3-(pyridin-4-yl)-4,5-dihydro-1H-benzo[g]indole-2-carboxylic acid;
51. 3-(3,5-difluorophenyl)-4,5-dihydro-1H-benzo[g]indole-2-carboxylic acid;
52. 3-(5-fluoropyridin-3-yl)-4,5-dihydro-1H-benzo[g]indole-2-carboxylic acid;
53. 3-(5-chloropyridin-3-yl)-4,5-dihydro-1H-benzo[g]indole-2-carboxylic acid;
54. 3-(2,3-dichlorophenyl)-4,5-dihydro-1H-benzo[g]indole-2-carboxylic acid;
55. 3-(pyrimidin-5-yl)-4,5-dihydro-1H-benzo[g]indole-2-carboxylic acid;
56. 3-(4-fluoro-2-methoxyphenyl)-4,5-dihydro-1H-benzo[g]indole-2-carboxylic acid;
57. 3-(3-chloro-5-methylphenyl)-4,5-dihydro-1H-benzo[g]indole-2-carboxylic acid;
58. 3-(3-chloro-5-fluorophenyl)-4,5-dihydro-1H-benzo[g]indole-2-carboxylic acid;
59. 3-(3-chloro-5-methoxyphenyl)-4,5-dihydro-1H-benzo[g]indole-2-carboxylic acid;
60. 3-(3,4,5-trichlorophenyl)-4,5-dihydro-1H-benzo[g]indole-2-carboxylic acid;
61. 3-(3,5-dimethoxyphenyl)-4,5-dihydro-1H-benzo[g]indole-2-carboxylic acid;
62. 3-(3-fluoro-5-methoxyphenyl)-4,5-dihydro-1H-benzo[g]indole-2-carboxylic acid;
63. 3-phenyl-4,5-dihydro-1H-benzo[g]indole-2-carboxylic acid;
64. 3-(3-chloro-5-fluorophenyl)-5-methyl-4,5-dihydro-1H-benzo[g]indole-2-carboxylic acid;
65. 7-bromo-3-(3,5-dichlorophenyl)-4,5-dihydro-1H-benzo[g]indole-2-carboxylic acid.

In an embodiment, present invention provides a process for the preparation of compound of formula I comprising the steps of:
i. reducing tetralone of formula (1) using reducing agents such as sodium borohydride to give compound of formula (2);

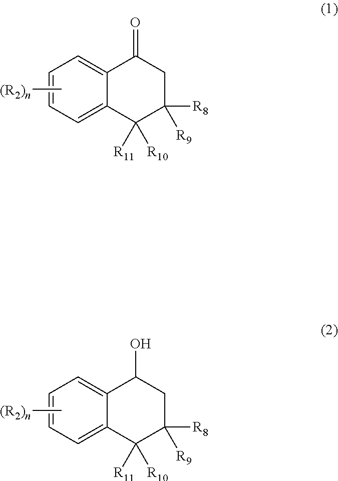

ii. formylation of compound of formula (2) as obtained in step (i) using in-situ prepared Vilsmeier Haack reagent/adduct to give compound of formula (3);

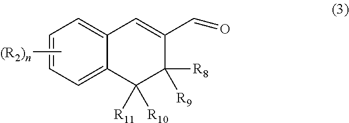

iii. treating aldehyde of formula (3) as obtained in step (ii) with azido ester compound of formula (4) in the presence of a base such as sodium ethoxide, sodium methoxide etc. to give compound of formula (5);

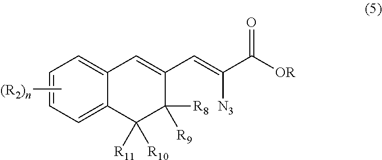

iv. cyclization of compound of formula (5) to get the compound of the formula (6) in the presence of suitable Lewis acid or in acidic condition;

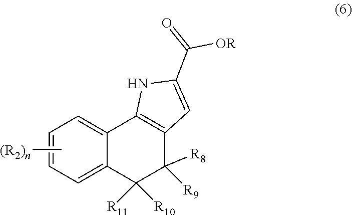

v. halogenating the compound of formula (6) using halogenated reagents such as NBS, NIS, etc. to give the halogenated compound of the formula (7);

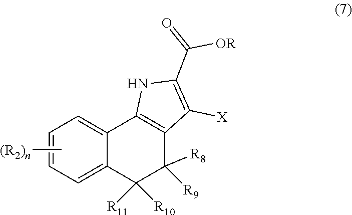

vi. coupling reaction of the halo compound of formula (7) with aryl/heteroaryl boronic acids or aryl/heteroaryl boronic esters to give compound of Formula (Ia);

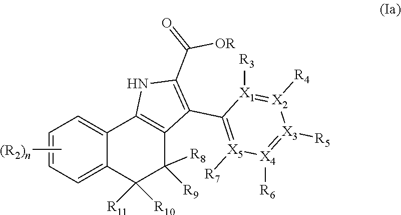

vii. converting compound of Formula (Ia) by hydrolysis of corresponding ester using base such as LiOH, K₂CO₃, NaOH to compound of Formula (I);

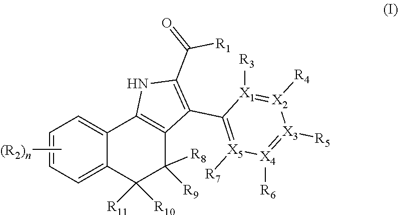

viii. In acid of formula (I) can be converted —COOH into corresponding amide, ester etc, wherein R1 is same as defined above.

In yet another embodiment of the present invention, compound of formula I in free form or in salt form or in pharmaceutically acceptable salt form.

In another embodiment, present invention provides a pharmaceutical composition comprising at least one compound of Formula (I) optionally along with pharmaceutically acceptable excipient.

In yet another embodiment of the present invention, said composition further comprises an effective amount of a beta-lactam antibiotic.

In yet another embodiment of the present invention, pharmaceutically acceptable excipients are selected from the group consisting of water, salt solutions, alcohols, polyethylene glycols, polyhydroxyethoxylated castor oil, peanut oil, olive oil, gelatin, lactose, terra alba, sucrose, dextrin, magnesium carbonate, sugar, cyclodextrin, amylose, magnesium stearate, talc, gelatin, agar, pectin, acacia, stearic acid or lower alkyl ethers of cellulose, salicylic acid, fatty acids, fatty acid amines, fatty acid monoglycerides and diglycerides, pentaerytritol fatty acid esters, polyoxyethylene, hydroxymethylcellulose and polyvinylpyrrolidone.

In yet another embodiment of the present invention, said composition is for use as a beta-lactamase inhibitor or as a drug.

In yet another embodiment of the present invention, use of a compound of formula (I), or a pharmaceutically acceptable salt thereof, for inhibiting beta-lactamase activity, in the manufacture of a medicament for inhibiting beta-lactamase activity, in combination with a beta-lactam antibiotic for treating a bacterial infection, or in combination with a beta-lactam antibiotic in the manufacture of a medicament for treating a bacterial infection.

In yet another embodiment, present invention provides a zwitterion of the compound of formula I.

### BRIEF DESCRIPTION OF THE DRAWING

FIG. 1 represents process steps for the preparation of compound of formula I.

### DETAILED DESCRIPTION OF THE INVENTION

Present invention provides substituted tricyclic heterocyclic compound of formula (I), pharmaceutically acceptable salts thereof.

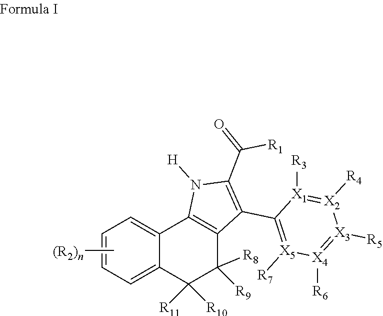

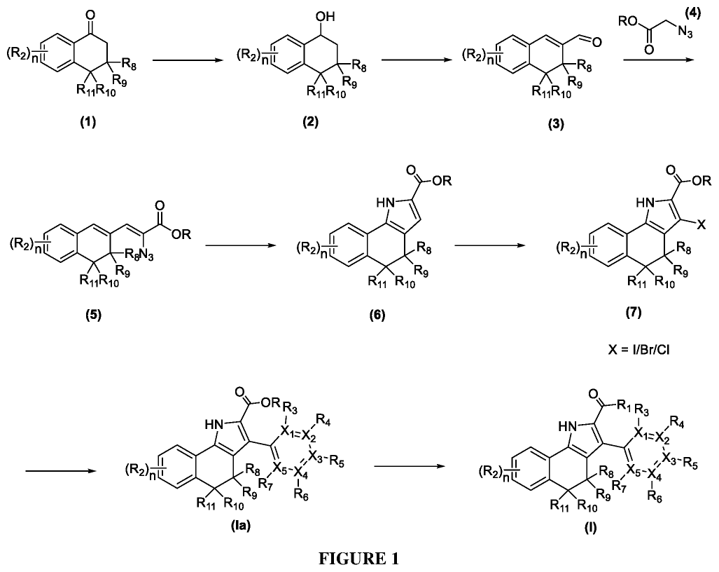

The compound of formula (I) where R₁, R₂, R₃, R₄, R₅, R₆, R₇, R₈, R₉, R₁₀, R₁₁, X₁, X₂, X₃, X₄, X₅ and ‘n’ are as defined herein above, can be prepared by following the procedure as depicted in FIG. 1 starting from commercially available tetralone of formula (1). Thus, tetralone of formula (1) undergoes reduction using suitable reducing agents such as sodium borohydride to give compound of formula (2). The compound of formula (2) undergoes formylation using in-situ prepared Vilsmeier Haack reagent/adduct to give compound of formula (3). Further, this aldehyde of formula (3) treated with azido ester compound of formula (4) in the presence of a base such as sodium ethoxide, sodium methoxide etc. to give compound of formula (5). The compound of formula (5) undergoes cyclization to get the compound of the formula (6) in the presence of suitable Lewis acid or in acidic condition. The compound of formula (6) undergoes halogenation using halogenated reagents such as NBS, NIS, etc. to give the halogenated compound of the formula (7). This halo compound of formula (7) undergoes coupling reaction with suitable aryl/heteroaryl boronic acids or aryl/heteroaryl boronic esters to give compound of Formula (Ia). Further, compound of Formula (Ia) is converted to compound Formula (I) by hydrolysis of corresponding ester using base such as LiOH, K₂CO₃, NaOH etc. In acid of formula (I) can be converted —COOH into corresponding amide, ester etc.

The terms “halogen” or “halo” means fluorine, chlorine, bromine, or iodine. The term “alkyl” refers to an alkane derived hydrocarbon radical that includes solely carbon and hydrogen atoms in the backbone, contains no unsaturation, has from one to six carbon atoms, and is attached to the remainder of the molecule by a single bond, for example C₁₋₄ alkyl or C₁₋₄ alkyl, representative groups include e.g., methyl, ethyl, n-propyl, l-methylethyl (isopropyl), n-butyl, n-pentyl and the like. Unless set forth or recited to the contrary, all alkyl groups described or claimed herein may be straight chain or branched.

The term “alkenyl” refers to a hydrocarbon radical containing from 2 to 10 carbon atoms and including at least one carbon-carbon double bond. Non-limiting Examples of alkenyl groups include, for example C₂₋₆ alkenyl, C₂₋₄ alkenyl, ethenyl, 1-propenyl, 2-propenyl (allyl), and the like. Unless set forth or recited to the contrary, all alkenyl groups described or claimed herein may be straight chain or branched.

The term “alkynyl” refers to a hydrocarbon radical containing 2 to 10 carbon atoms and including at least one carbon-carbon triple bond. Non-limiting Examples of alkynyl groups include, for example C₂₋₆ alkynyl, C₂₋₄ alkynyl, ethynyl, propynyl, butynyl and the like. Unless set forth or recited to the contrary, all alkynyl groups described or claimed herein may be straight chain or branched.

The term “haloalkyl” refers to an alkyl group as defined above that is substituted by one or more halogen atoms as defined above. For example C₁₋₆haloalkyl or C₁₋₄ haloalkyl. Suitably, the haloalkyl may be monohaloalkyl, dihaloalkyl or polyhaloalkyl including perhaloalkyl. A monohaloalkyl can have one iodine, bromine, chlorine or fluorine atom. Dihaloalkyl and polyhaloalkyl groups can be substituted with two or more of the same halogen atoms or a combination of different halogen atoms. Suitably, a polyhaloalkyl is substituted with up to 12 halogen atoms. Non-limiting Examples of a haloalkyl include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl, dichloropropyl and the like. A perhaloalkyl refers to an alkyl having all hydrogen atoms replaced with halogen atoms. Unless set forth or recited to the contrary, all haloalkyl groups described or claimed herein may be straight chain or branched.

The term “alkoxy” denotes an alkyl group attached via an oxygen linkage to the rest of the molecule. Representative examples of such groups are —OCH₃ and —OC₂H₅. Unless set forth or recited to the contrary, all alkoxy groups described or claimed herein may be straight chain or branched.

The term “alkoxyalkyl” refers to an alkoxy group as defined above directly bonded to an alkyl group as defined above, e.g., —CH₂—O—CH₃, —CH₂—O—CH₂CH₃, —CH₂CH₂—O—CH₃ and the like.

The term “cycloalkyl” refers to a non-aromatic mono or multicyclic ring system having 3 to 12 carbon atoms, such as C₃₋₁₀ cycloalkyl, C₃₋₆ cycloalkyl, cyclopropyl, cyclobutyl, cyclopentyl, cyclohexyl and the like. Examples of multicyclic cycloalkyl groups include, but are not limited to, perhydronaphththyl, adamantyl and norbornyl groups, bridged cyclic groups or Spiro bicyclic groups, e.g., spiro(4,4)non-2-yl and the like.

The term “aryl” refers to an aromatic radical having 6- to 14-carbon atoms, including monocyclic, bicyclic and tricyclic aromatic systems, such as phenyl, naphthyl, tetrahydronaphthyl, indanyl, and biphenyl and the like.

The term “heterocyclic ring” or “heterocyclyl ring” or “heterocyclyl”, unless otherwise specified, refers to substituted or unsubstituted non-aromatic 3- to 15-membered ring which consists of carbon atoms and with one or more heteroatom(s) independently selected from N, O or S. The heterocyclic ring may be a mono-, bi- or tricyclic ring system, which may include fused, bridged or spiro ring systems and the nitrogen, carbon, oxygen or sulfur atoms in the heterocyclic ring may be optionally oxidized to various oxidation states. In addition, the nitrogen atom may be optionally quaternized, the heterocyclic ring or heterocyclyl may optionally contain one or more olefinic bond, and one or two carbon atoms/in the heterocyclic ring or heterocyclyl may be interrupted with —CF₂—, —C(O)—, —S(O)—, S(O)₂ etc. In addition, heterocyclic ring may also be fused with aromatic ring. Non-limiting Examples of heterocyclic rings include azetidinyl, benzopyranyl, chromanyl, decahydroisoquinolyl, indolinyl, isoindolinyl, isochromanyl, isothiazolidinyl, isoxazolidinyl, morpholinyl, oxazolinyl, oxazolidinyl, 2-oxopiperazinyl, 2-oxopiperidinyl, 2-oxopyrrolidinyl, 2-oxoazepinyl, octahydroindolyl, octahydroisoindolyl, perhydroazepinyl, piperazinyl, 4-piperidonyl, pyrrolidinyl, piperidinyl, phenothiazinyl, phenoxazinyl, quinuclidinyl, tetrahydroisquinolyl, tetrahydrofuryl, tetrahydropyranyl, thiazolinyl, thiazolidinyl, thiamorpholinyl, thiamorpholinylsulfoxide, thiamorpholinylsulfoneindoline, benzodioxole, tetrahydroquinoline, tetrahydrobenzopyran and the like. The heterocyclic ring may be attached by any atom of the heterocyclic ring that results in the creation of a stable structure.

The term “heteroaryl” unless otherwise specified, refers to a substituted or unsubstituted 5- to 14-membered aromatic heterocyclic ring with one or more heteroatom/s independently selected from N, O or S. The heteroaryl may be a mono-, bi- or tricyclic ring system. The heteroaryl ring may be attached by any atom of the heteroaryl ring that results in the creation of a stable structure. Non-limiting Examples of a heteroaryl ring include oxazolyl, isoxazolyl, imidazolyl, furyl, indolyl, isoindolyl, pyrrolyl, triazolyl, triazinyl, tetrazolyl, thienyl, thiazolyl, isothiazolyl, pyridyl, pyrimidinyl, pyrazinyl, pyridazinyl, benzofuranyl, benzothiazolyl, benzoxazolyl, benzimidazolyl, benzothienyl, carbazolyl, quinolinyl, isoquinolinyl, quinazolinyl, cinnolinyl, naphthyridinyl, pteridinyl, purinyl, quinoxalinyl, quinolyl, isoquinolyl, thiadiazolyl, indolizinyl, acridinyl, phenazinyl, phthalazinyl and the like.

The term “treating” or “treatment” of a state, disorder or condition includes: (a) preventing or delaying the appearance of clinical symptoms of the state, disorder or condition developing in a subject that may be afflicted with or predisposed to the state, disorder or condition but does not yet experience or display clinical or subclinical symptoms of the state, disorder or condition; (b) inhibiting the state, disorder or condition, i.e., arresting or reducing the development of the disease or at least one clinical or subclinical symptom thereof; c) lessening the disease, disorder or condition or at least one of its clinical or subclinical symptoms or (d) relieving the disease, i.e., causing regression of the state, disorder or condition or at least one of its clinical or subclinical symptoms.

The term “inhibitor” refers to a molecule that binds to an enzyme to inhibit the activity of the said enzyme either partially or completely.

The term “effective amount” refers to the amount of each active agent required to confer the desired effect (e.g., inhibiting MBL) on the subject, either alone or in combination with one or more other active agents. An effective amount varies, as recognized by those skilled in the art, depending on the particular condition being treated, the severity of the condition, the individual patient parameters including age, size, physical condition, weight, and gender, the nature of concurrent therapy (if any), the duration of the treatment, the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

A “therapeutically effective amount” means the amount of a compound that, when administered to a subject for treating a disease, disorder or condition, is sufficient to cause the effect in the subject, which is the purpose of the administration. The “therapeutically effective amount” will vary depending on the compound, the disease and its severity and the age, weight, physical condition and responsiveness of the subject to be treated.

### Pharmaceutically Acceptable Salts

The compounds of the invention may form salts with acid or base. The compounds of invention may be sufficiently basic or acidic to form stable nontoxic acid or base salts, administration of the compound as a pharmaceutically acceptable salt may be appropriate. Non-limiting Examples of pharmaceutically acceptable salts are inorganic, organic acid addition salts formed by addition of acids including hydrochloride salts. Non-limiting Examples of pharmaceutically acceptable salts are inorganic, organic base addition salts formed by addition of bases. The compounds of the invention may also form salts with amino acids. Pharmaceutically acceptable salts may be obtained using standard procedures well known in the art, for example by reacting sufficiently basic compound such as an amine with a suitable acid.

Screening of the compounds of invention for MBL inhibitory activity can be achieved by using various in-vitro mentioned herein below or methods known in the art.

### Pharmaceutical Compositions

The invention relates to pharmaceutical compositions containing the compounds of the formula (I), or pharmaceutically acceptable salts thereof disclosed herein. In particular, pharmaceutical compositions containing a therapeutically effective amount of at least one compound of formula (I) described herein and at least one pharmaceutically acceptable excipient (such as a carrier or diluent). Preferably, the contemplated pharmaceutical compositions include the compound(s) described herein in an amount sufficient to inhibit MBL to treat the diseases described herein when administered to a subject.

The subjects contemplated include, for example, a living cell and a mammal, including human. The compound of the invention may be associated with a pharmaceutically acceptable excipient (such as a carrier or a diluent) or be diluted by a carrier, or enclosed within a carrier which can be in the form of a capsule, sachet, paper or other container. The pharmaceutically acceptable excipient includes pharmaceutical agent that does not itself induce the production of antibodies harmful to the individual receiving the composition, and which may be administered without undue toxicity.

Examples of suitable carriers or excipients include, but are not limited to, water, salt solutions, alcohols, polyethylene glycols, polyhydroxyethoxylated castor oil, peanut oil, olive oil, gelatin, lactose, terra alba, sucrose, dextrin, magnesium carbonate, sugar, cyclodextrin, amylose, magnesium stearate, talc, gelatin, agar, pectin, acacia, stearic acid or lower alkyl ethers of cellulose, salicylic acid, fatty acids, fatty acid amines, fatty acid monoglycerides and diglycerides, pentaerytritol fatty acid esters, polyoxyethylene, hydroxymethylcellulose and polyvinylpyrrolidone.

The pharmaceutical composition may also include one or more pharmaceutically acceptable auxiliary agents, wetting agents, emulsifying agents, suspending agents, preserving agents, salts for influencing osmotic pressure, buffers, sweetening agents, flavouring agents, colorants, or any combination of the foregoing. The pharmaceutical composition of the invention may be formulated so as to provide quick, sustained, or delayed release of the active ingredient after administration to the subject by employing procedures known in the art.

The pharmaceutical compositions described herein may be prepared by conventional techniques known in the art. For example, the active compound can be mixed with a carrier, or diluted by a carrier, or enclosed within a carrier, which may be in the form of an ampoule, capsule, sachet, paper, or other container. When the carrier serves as a diluent, it may be a solid, semi-solid, or liquid material that acts as a vehicle, excipient, or medium for the active compound. The active compound can be adsorbed on a granular solid container, for Example, in a sachet. The pharmaceutical compositions may be in conventional forms, for example, capsules, tablets, caplets, orally disintegrating tablets, aerosols, solutions, suspensions or products for topical application.

The route of administration may be any route which effectively transports the active compound of the invention to the appropriate or desired site of action. Suitable routes of administration include, but are not limited to, oral, oral inhalation, nasal, pulmonary, buccal, subdermal, intradermal, transdermal, parenteral, rectal, depot, subcutaneous, intravenous, intraurethral, intramuscular, intranasal, ophthalmic (such as with an ophthalmic solution) or topical (such as with a topical ointment).

Solid oral formulations include, but are not limited to, tablets, caplets, capsules (soft or hard gelatin), orally disintegrating tablets, dragees (containing the active ingredient in powder or pellet form), troches and lozenges. Tablets, dragees, or capsules having talc and/or a carbohydrate carrier or binder or the like are particularly suitable for oral application. Liquid formulations include, but are not limited to, syrups, emulsions, suspensions, solutions, soft gelatin and sterile injectable liquids, such as aqueous or non-aqueous liquid suspensions or solutions. For parenteral application, particularly suitable are injectable solutions or suspensions, preferably aqueous solutions with the active compound dissolved in polyhydroxylated castor oil.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as pocketed tablets, capsules, and powders in vials or ampoules. Also, the unit dosage form can be a capsule, tablet, caplet, cachet, or lozenge itself, or it can be the appropriate number of any of these in packaged form.

For administration to subject patients, the total daily dose of the compounds of the invention depends, of course, on the mode of administration. For example, oral administration may require a higher total daily dose, than an intravenous (direct into blood). The quantity of active component in a unit dose preparation may be varied or adjusted from 0.1 mg to 1000 mg by oral administration and 1 pg to 5000 pg by inhalation according to the potency of the active component or mode of administration.

Those skilled in the relevant art can determine suitable doses of the compounds for use in treating the diseases and disorders described herein. Therapeutic doses are generally identified through a dose ranging study in subject based on preliminary evidence derived from the animal studies. Doses must be sufficient to result in a desired therapeutic benefit without causing unwanted side effects for the patient. For example, the daily dosage of the MBL inhibitor can range from about 0.1 to about 30.0 mg/kg by oral administration. Mode of administration, dosage forms, suitable pharmaceutical excipients, diluents or carriers can also be well used and adjusted by those skilled in the art. All changes and modifications envisioned are within the scope of the invention.

### Methods of Treatment

The invention provides compound of formula (I) and pharmaceutical compositions thereof as MBL inhibitors for treating the diseases, disorders or conditions associated with antimicrobial resistance (AMR). The invention further provides a method of treating diseases, disorders or conditions associated with antimicrobial resistance in a subject in need thereof by administering to the subject a therapeutically effective amount of a compound or a pharmaceutical composition of the invention.

In another aspect, the invention relates to a method of treating diseases, disorders or conditions associated with MBL, AMR, antibacterial. In this method, a subject in need of such treatment is administered a therapeutically effective amount of a compound of formula (I) or a pharmaceutically acceptable salt thereof as described herein. Present invention encompasses the compounds of formula (I) or pharmaceutically acceptable salts thereof in the manufacture of a medicament for treating a disease or disorder mentioned herein.

### EXAMPLES

Following examples are given by way of illustration and therefore should not be construed to limit the scope of the invention.

### General Procedure for Preparation of Intermediate 1

### Methyl 3-iodo-4,5-dihydro-1H-benzo[g]indole-2-carboxylate

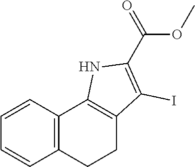 [SMILES: COC(=O)c1[nH]c2c(c1I)CCc1ccccc1-2]

### Step 1: Preparation of 1,2,3,4-tetrahydronaphthalen-1-ol

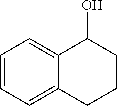 [SMILES: OC1CCCc2ccccc21]

NaBH₄ (3.88 g, 102.61 mmol) was added to a stirred solution of 1-tetralone (15 g, 102.61 mmol) in methanol (256 mL). The reaction mixture was stirred for 1 hour and then concentrated to about 35 mL under reduced pressure. To this reaction mixture, water was added (75 mL) and extracted with ethyl acetate (2×100 mL). The organic phase was washed with brine solution (40 mL), dried over Na₂SO₄ and filtered. The organic layer was concentrated to get crude compound. The crude compound was purified by flash column chromatography (Biotage) using eluent (1:9, ethyl acetate:n-hexane) to get the titled compound (15.1 g, 99.3% yield) as a pale yellow oil. LCMS (ESI): m/z=148.2 (M)⁺

### Step 2: Preparation of 3,4-dihydronaphthalene-2-carbaldehyde

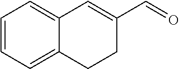 [SMILES: O=CC1=Cc2ccccc2CC1]

To a stirred solution of 1,2,3,4-tetrahydronaphthalen-1-ol (15.4 g, 97.5 mmol) in DMF (39.22 mL), POCl₃ (22.66 mL) was added dropwise at 0° C. and stirred for one hour. The reaction mixture was allowed to rise at room temperature and stirred for another one hour. Further, the stirred solution was heated at 100° C. for overnight. The reaction mixture was quenched at room temperature with water and basified with 3M NaOH. The reaction mixture was extracted with ethyl acetate (3×550 mL), washed with brine solution, dried over Na₂SO₄, and concentrated to get the crude compound. The crude compound was purified by flash column chromatography (Biotage) using eluent (1:9, ethyl acetate:n-hexane) to get the 3,4-dihydronaphthalene-2-carbaldehyde (8.5 g, 52% yield) as a pale yellow oil.

¹H NMR (300 MHz, DMSO-D₆) δ=9.62 (s, 1H), 7.51 (s, 1H), 7.37-7.24 (m, 4H), 2.79 (t, J=8.58 Hz, 2H), 2.42 (t, J=8.58 Hz, 2H).

### Step 3: Preparation of methyl (Z)-2-azido-3-(3,4-dihydronaphthalen-2-yl) acrylate

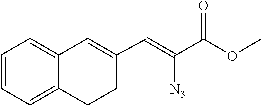 [SMILES: COC(=O)/C(=C/C1=Cc2ccccc2CC1)N=[N+]=[N-]]

A round bottomed flask purged and maintained with an inert atmosphere was charged with 3,4-dihydronaphthalene-2-carbaldehyde (8.5 g, 53.95 mmol), methyl azidoacetate (15.5 g, 134.18 mmol, 1.18 mL) in dry MeOH (175 mL) at −15° C. After 15 minutes to the stirred mixture, a solution of NaOMe (26.34 mL) in MeOH (44 mL) was added dropwise over 20 min and stirred at −15° C. for 90 min. Further, slowly warmed to 4° C. and stirred for 12 h. The reaction mixture was then poured into ice-cold saturated aqueous NH₄Cl (215 mL). The resulting precipitate was isolated on a fritted funnel and washed with deionized water until the filtrate came through clear. The solid was dissolved in DCM and dried over Na₂SO₄. The organic phase was filtered and evaporated in vacuo to get crude compound. The crude compound was purified by flash column chromatography (Biotage) using eluent (1:9, ethyl acetate:n-hexane) to get the methyl (Z)-2-azido-3-(3,4-dihydronaphthalen-2-yl) acrylate (9.65 g, 70% yield) as an oil with trace cyclized compound, which was further taken for next reaction.

### Step 4: Preparation of methyl 4,5-dihydro-1H-benzo[g]indole-2-carboxylate

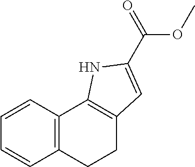 [SMILES: COC(=O)c1cc2c([nH]1)-c1ccccc1CC2]

To a stirred solution of methyl 4,5-dihydro-1H-benzo[g]indole-2-carboxylate (9.654 g, 37.82 mmol) in DCM (55 mL), ZnI₂ (0.604 g, 1.89 mmol) was added slowly and stirred overnight at room temperature. The resulting solution was filtered through a pad of Celite and concentrated under vacuum to get the crude compound. The crude compound was purified by flash column chromatography (Biotage) using eluent (2:8, ethyl acetate:n-hexane) to get the methyl 4,5-dihydro-1H-benzo[g]indole-2-carboxylate (5.86 g, 68% yield) as a pale yellow solid.

LCMS (ESI): m/z=228.1 (M+H)⁺; ¹H NMR (300 MHz, DMSO-D₆) δ=12.08 (s, 1H), 7.84 (d, J=7.6 Hz, 1H), 7.18 (t, J=7.5 Hz, 2H), 7.13-7.07 (m, 1H), 6.70-6.62 (m, 1H), 3.75 (s, 3H), 2.82 (t, J=7.5 Hz, 2H), 2.62 (dd, J=8.5, 6.5 Hz, 2H).

### Step 5: Preparation of methyl 3-iodo-4,5-dihydro-1H-benzo[g]indole-2-carboxylate

To a stirred solution of methyl 3-iodo-4,5-dihydro-1H-benzo[g]indole-2-carboxylate (5.86 g, 25.8 mmol) in DMF (60 mL) was added N-iodosuccinamide (6.38 g, 28.34 mmol). The resulting mixture was stirred at room temperature for 3 h and then concentrated in vacuo. The crude mixture was dissolved in DCM and washed with sat. NaHCO₃. The organic extracts were dried over Na₂SO₄, filtered and concentrated in vacuo. The crude compound was purified by column chromatography (Biotage) using eluent (2:8, ethyl acetate:n-hexane) to get the methyl 3-iodo-4,5-dihydro-1H-benzo[g]indole-2-carboxylate (8.01 g, 88% yield) as a brown solid. LCMS (ESI): m/z=354.1 (M+H)⁺; ¹H NMR (300 MHz, DMSO-D₆) δ=12.42 (s, 1H), 7.89 (d, J=7.5 Hz, 1H), 7.35-6.99 (m, 3H), 3.79 (s, 3H), 2.87 (t, J=7.6 Hz, 2H), 2.56-2.44 (m, 2H).

### Example 1: Preparation of methyl 3-(3,5-dichloropbenyl)-4,5-dihydro-1H-benzo[g]indole-2-carboxylate

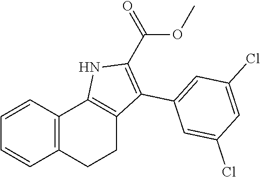 [SMILES: COC(=O)c1[nH]c2c(c1-c1cc(Cl)cc(Cl)c1)CCc1ccccc1-2]

To a solution of methyl 3-iodo-4,5-dihydro-1H-benzo[g]indole-2-carboxylate (200 mg, 0.57 mmol), 3,5-dichlorophenylboronic acid (130 g, 0.68 mmol) in 1,4-dioxane (5.5 mL), aqueous Na₂CO₃ (120.8 mg, 1.14 mmol in 1.5 mL H₂O) was added at 25° C. in a sealed tube and nitrogen gas was bubbled through the reaction mixture for 15 minutes. To this [1,1′-Bis(diphenylphosphino)ferrocene]dichloropalladium(II) was added, packed tightly and the reaction mixture was heated with stirring at 80° C. for 16 h. The progress of the reaction was monitored by TLC. The reaction mixture was cooled to 25° C. and filtered through celite. The filtrate was concentrated under reduced pressure to obtain a crude product. The crude compound so obtained was purified by column chromatography (Biotage) using eluent (2:8, ethyl acetate:n-hexane) to get the methyl 3-(3,5-dichlorophenyl)-4,5-dihydro-1H-benzo[g]indole-2-carboxylate (188 mg, 89% yield) as a solid.

LCMS (ESI): m/z=370.0, 372 (M-2H)⁻; ¹H NMR (300 MHz, DMSO-D₆) δ=12.08 (s, 1H), 7.93 (d, J=7.2 Hz, 1H), 7.51 (s, 1H), 7.38 (s, 2H), 7.23-7.20 (m, 2H), 7.18-7.13 (m, 1H), 3.68 (s, 3H), 2.83 (t, J=7.23 Hz, 2H),
2.56-2.51 (m, 2H).

### Example 2: Preparation of 3-(3,5-dichlorophenyl)-4,5-dihydro-1H-benzo[g]indole-2-carboxylic Acid

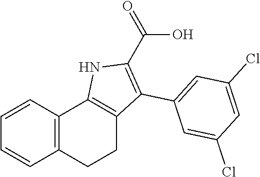 [SMILES: O=C(O)c1[nH]c2c(c1-c1cc(Cl)cc(Cl)c1)CCc1ccccc1-2]

To a stirred solution of methyl 3-(3,5-dichlorophenyl)-4,5-dihydro-1H-benzo[g]indole-2-carboxylate (188 mg, 0.57 mmol) in THF (2.5 mL), MeOH (1.1 mL) and H₂O (1.1 mL) was added LiOH·H₂O (120 mg, 2.87 mmol). The resultant mixture was stirred at room temperature for 48 h. Reaction mixture was acidified to pH 2 with 2 M HCl and extracted with EtOAc (2×25 mL). The organic extracts were dried over Na₂SO₄, filtered and concentrated in vacuo. The crude compound so obtained was purified by column chromatography (Biotage) using eluent (2:8, ethyl acetate:n-hexane) to get the 3-(3,5-dichlorophenyl)-4,5-dihydro-1H-benzo[g]indole-2-carboxylic acid (112 g, 60% yield) as a solid.

LCMS (ESI): m/z=356.3 (M-2H)⁻; ¹H NMR (300 MHz, CHLOROFORM-D) δ=9.37 (bs, 1H), 7.46-7.13 (m, 7H), 2.94 (t, J=7.2 Hz, 2H), 2.66 (t, J=7.2 Hz, 2H).

### Example 3: Preparation of methyl 3-(5-fluoro-2-methoxyphenyl)-4,5-dihydro-1H-benzo[g]indole-2-carboxylate

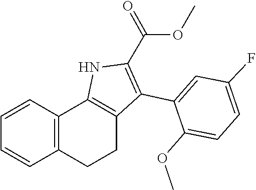 [SMILES: COC(=O)c1[nH]c2c(c1-c1cc(F)ccc1OC)CCc1ccccc1-2]

Following a procedure analogous to the one provided for compound of example 1 and replacing 3,5-dichlorophenylboronic acid with an appropriate boronic acid.

LCMS (ESI): m/z=352.1 (M+H)⁺; ¹H NMR (300 MHz, CHLOROFORM-D) δ=9.33 (bs, 1H), 7.39 (d, J=7.6 Hz, 1H), 7.29-7.14 (m, 3H), 7.05-6.96 (m, 2H), 6.91-6.84 (m, 1H), 3.75 (s, 3H), 3.73 (s, 3H), 2.91 (t, J=7.5 Hz, 2H), 2.51-2.60 (m, 2H).

### Example 4: Preparation of methyl 3-(2-methoxyphenyl)-4,5-dihydro-1H-benzo[g]indole-2-carboxylate

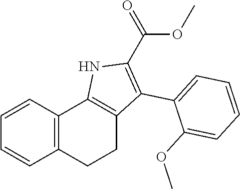 [SMILES: COC(=O)c1[nH]c2c(c1-c1ccccc1OC)CCc1ccccc1-2]

Following a procedure analogous to the one provided for compound of example 1 and replacing 3,5-dichlorophenylboronic acid with an appropriate boronic acid.

LCMS (ESI): m/z=334.1 (M+H)⁺; ¹H NMR (300 MHz, CHLOROFORM-D) δ=9.30 (bs, 1H), 7.49-7.10 (m, 4H), 7.08-6.90 (m, 4H), 3.78 (s, 3H), 3.71 (s, 3H), 2.91 (t, J=7.6 Hz, 2H), 2.61-2.57 (m 2H).

### Example 5: Preparation of methyl 3-(4-methoxyphenyl)-4,5-dihydro-1H-benzo[g]indole-2-carboxylate

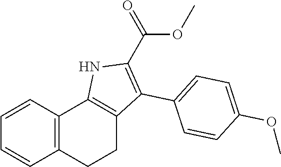 [SMILES: COC(=O)c1[nH]c2c(c1-c1ccc(OC)cc1)CCc1ccccc1-2]

Following a procedure analogous to the one provided for compound of example 1 and replacing 3,5-dichlorophenylboronic acid with an appropriate boronic acid.

LCMS (ESI): m/z=334.1 (M+H)⁺; ¹H NMR (300 MHz, CHLOROFORM-D) δ=9.20 (bs, 1H), 7.47−7.26 (m, 3H), 7.24-7.05 (m,3H), 7.05-6.86 (d, 2H), 3.89 (s, 3H), 3.76 (s, 3H), .92 (t, J=7.9 Hz 2H),2.69-2.67 (m, 2H).

### Example 6: Preparation of methyl 3-(3,4difluorophenyl-4,5-dihydro-1H-benzo[g]indole-2-carboxylate

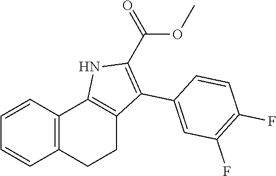 [SMILES: COC(=O)c1[nH]c2c(c1-c1ccc(F)c(F)c1)CCc1ccccc1-2]

Following a procedure analogous to the one provided for compound of example 1 and replacing 3,5-dichlorophenylboronic acid with an appropriate boronic acid.

LCMS (ESI): m/z=338.1 (M+H)⁺; ¹H NMR (300 MHz, CHLOROFORM-D) δ=9.27 (bs, 1H), 7.37-7.34 (m, 2H), 7.21 (dd, J=10.3, 4.5 Hz, 2H), 6.99-6.88 (m, 2H), 6.81-6.74 (m, 1H), 3.78 (s, 3H), 2.93 (t, 2H), 2.67 (t, 2H).

### Example 7: Preparation of methyl 3-(5-chloro-2-methoxphenyl)-4,5-dihydro-1H-benzo[g]indole-2-carboxylate

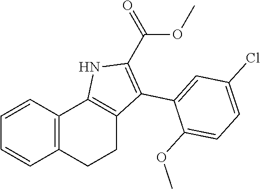 [SMILES: COC(=O)c1[nH]c2c(c1-c1cc(Cl)ccc1OC)CCc1ccccc1-2]

Following a procedure analogous to the one provided for compound of example 1 and replacing 3,5-dichlorophenylboronic acid with an appropriate boronic acid.

LCMS (ESI): m/z=368.0 (M+H)⁺; ¹H NMR (300 MHz, CHLOROFORM-D) δ=9.25 (bs, 1H), 7.37 (d, J=7.4 Hz, 1H), 7.28 (dd, J=4.9, 2.4 Hz, 1H), 7.25-7.13 (m, 4H), 6.89 (d, J=8.5 Hz 1H), 3.75 (s, 3H), 3.72 (s, 3H), 2.91 (t, J=7.5 Hz, 2H), 2.61-2.51 (m, 2H).

### Example 8: Preparation of methyl 3-(4-(tert-butyl)phenyl)-4,5-dihydro-1H-benzo[g]indole-2-carboxylate

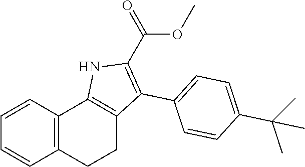 [SMILES: COC(=O)c1[nH]c2c(c1-c1ccc(C(C)(C)C)cc1)CCc1ccccc1-2]

Following a procedure analogous to the one provided for compound of example 1 and replacing 3,5-dichlorophenylboronic acid with an appropriate boronic acid.

LCMS (ESI): m/z=360.2 (M+H)⁺; ¹H NMR (300 MHz, CHLOROFORM-D) δ=9.23 (bs, 1H), 7.45-7.34 (m, 5H), 7.25-7.15 (m, 3H), 3.77 (s, 3H), 2.91 (t, J=7.8 Hz, 2H), 2.72 (t, J=7.8 Hz, 2H), 1.36 (s, 9H).

### Example 9: Preparation of methyl 3-(4-fluorophenyl)4,5-dihydro-1H-benzo[g]indole-2-carboxylate

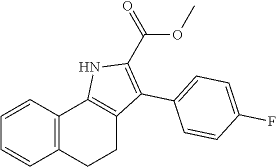 [SMILES: COC(=O)c1[nH]c2c(c1-c1ccc(F)cc1)CCc1ccccc1-2]

Following a procedure analogous to the one provided for compound of example 1 and replacing 3,5-dichlorophenylboronic acid with an appropriate boronic acid.

LCMS (ESI): m/z=322.1 (M+H)⁺; 1H NMR (300 MHz, CHLOROFORM-D) δ=9.39-9.12 (bs, 1H), 7.38 (dd, J=8.9, 5.7 Hz, 3H), 7.25-7.14 (m, 3H), 7.09 (t, J=8.9 Hz, 2H), 3.76 (s, 3H), 2.93 (t, J=7.8 Hz, 2H), 2.67 (t, J=7.8 Hz, 2H).

### Example 10: Preparation of methyl 3-phenyl-4,5-dihydro-1H-benzo[g]indole-2-carboxylate

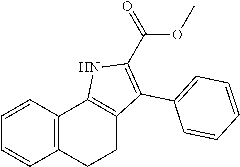 [SMILES: COC(=O)c1[nH]c2c(c1-c1ccccc1)CCc1ccccc1-2]

Following a procedure analogous to the one provided tor compound of example-1 and replacing 3,5-dichlorophenylboronic acid with an appropriate boronic acid.

LCMS (ESI): m/z=304.1 (M+H)⁺; ¹H NMR (300 MHz, CHLOROFORM-D) δ=7.55-7.26 (m, 5H), 7.25-7.22 (m, 4H), 3.76 (s, 3H), 2.92 (t, J=7.8 Hz, 2H), 2.68 (t, J=7.8 Hz, 2H).

### Example 11: Preparation of methyl 3-(2-fluorophenyl)-4,5-dihydro-1H-benzo[g]indole-2-carboxylate

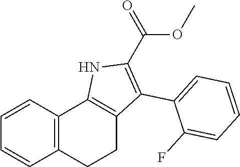 [SMILES: COC(=O)c1[nH]c2c(c1-c1ccccc1F)CCc1ccccc1-2]

Following a procedure analogous to the one provided for compound of example 1 and replacing 3,5-dichlorophenylboronic acid with an appropriate boronic acid.

LCMS (ESI): m/z=322.1 (M+H)+; ¹H NMR (500 MHz, DMSO) δ=12.27 (s, 1H), 8.04-7.87 (m, 1H), 7.42-7.31 (m, 2H), 7.29-7.20 (m, 4H), 7.20-7.13 (m, 1H), 3.65 (s, 3H), 2.85 (t, J=7.5 Hz, 2H), 2.58-2.39 (m, 2H).

### Example 12: Preparation of methyl 3-(3-fluorophenyl)-4,5-dihydro-1H-benzo[g]indole-2-carboxylate

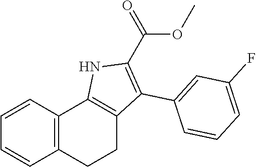 [SMILES: COC(=O)c1[nH]c2c(c1-c1cccc(F)c1)CCc1ccccc1-2]

Following a procedure analogous to the one provided for compound of example 1 and replacing 3,5-dichlorophenylboronic acid with an appropriate boronic acid.

LCMS (ESI): m/z=322.1 (M+H)⁺; ¹H NMR (500 MHz, DMSO) δ=12.19 (s, 1H), 8.03-7.91 (m, 1H), 7.47-7.37 (m, 1H), 7.31-7.22 (m, 2H), 7.17-7.09 (m, 4H), 3.69 (s, 3H), 2.85 (t, J=7.5 Hz, 2H), 2.53 (m, 2H).

### Example 13: Preparation of methyl 3-(4-chlorophenyl)-4,5-dihydro-1H-benzo[g]indole-2-carboxylate

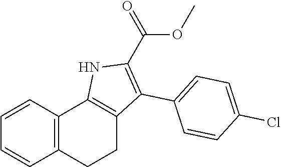 [SMILES: COC(=O)c1[nH]c2c(c1-c1ccc(Cl)cc1)CCc1ccccc1-2]

Following a procedure analogous to the one provided for compound of example 1 and replacing 3,5-dichlorophenylboronic acid with an appropriate boronic acid

LCMS (ESI): m/z=338.1 (M+H)⁺; ¹H NMR (500 MHz, DMSO) δ=12.37-11.94 (m, 1H), 7.97 (d, J=7.1 Hz, 1H), 7.48-7.41 (m, 2H), 7.41-7.36 (m, 2H), 7.25 (d, J=2.7 Hz, 2H), 7.17 (d, J=0.9 Hz, 1H), 3.68 (s, 3H), 2.84 (t, J=7.7 Hz, 2H), 2.67-2.44 (m, 2H).

### Example 14: Preparation of methyl 3-(2-chlorophenyl)-4,5-dihydro-1H-benzo[g]indole-2-carboxylate

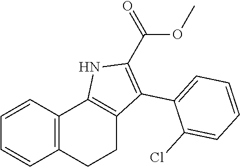 [SMILES: COC(=O)c1[nH]c2c(c1-c1ccccc1Cl)CCc1ccccc1-2]

Following a procedure analogous to the one provided for compound of example 1 and replacing 3,5-dichlorophenylboronic acid with an appropriate boronic acid LCMS (ESI): m/z=338.1 (M+H)⁺; ¹H NMR (500 MHz, DMSO)δ=12.24 (bs, 1H), 7.96 (d, J=7.7 Hz, 1H), 7.51 (dd, J=5.7, 3.2 Hz, 1H), 7.40-7.33 (m, 3H), 7.24 (d, J=7.5 Hz, 2H), 7.16-7.07 (m, 1H), 3.61 (s, 3H), 2.84 (t, J=7.5 Hz, 2H), 2.46-2.31 (m, 2H).

### Example 15: Preparation of methyl 3-(3-chlorophenyl)-4,5-dihydro-1H-benzo[g]indole-2-carboxylate

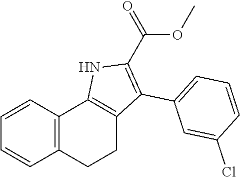 [SMILES: COC(=O)c1[nH]c2c(c1-c1cccc(Cl)c1)CCc1ccccc1-2]

Following a procedure analogous to the one provided for compound of example 1 and replacing 3,5-dichlorophenylboronic acid with an appropriate boronic acid LCMS (ESI): m/z=338.1 (M+H)⁺; ¹H NMR (500 MHz, DMSO) δ=12.21 (s, 1H), 8.05-7.91 (m, 1H), 7.44-7.40 (m, 2H), 7.38 (dd, J=6.0, 4.0 Hz, 1H), 7.34-7.30 (m, 1H), 7.28-7.23 (m, 2H), 7.18 (dd, J=7.3, 1.1 Hz, 1H), 3.69 (s, 3H), 2.85 (t, J=7.5 Hz, 2H), 2.63-2.51 (m, 2H).

### Example 16: Preparation of Methyl 3-(2,4-dichlorophenyl)-4,5-dihydro-1H-benzo[g]indole-2-carboxylate

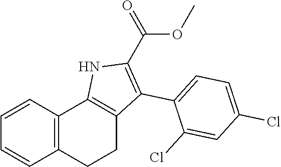 [SMILES: COC(=O)c1[nH]c2c(c1-c1ccc(Cl)cc1Cl)CCc1ccccc1-2]

Following a procedure analogous to the one provided for compound of example 1 and replacing 3,5-dichlorophenylboronic acid with an appropriate boronic acid

LCMS (ESI): m/z=374.0 (M+H)⁺; ¹H NMR (500 MHz, DMSO) δ=12.39 (bs, 1H), 7.95 (s, 1H), 7.68 (d, J=2.1 Hz, 1H), 7.43 (d, J=2.1 Hz, 1H), 7.38 (d, J=8.3 Hz, 1H), 7.25 (d, J=7.4 Hz, 2H), 7.17 (s, 1H), 3.63 (s, 3H), 2.85 (m, 2H), 2.45 (m, 2H).

### Example 17: Preparation of methyl 3-(2,3-difluorophenyl)-4,5-dihydro-1H-benzo[g]indole-2-carboxylate

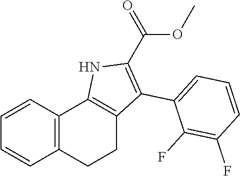 [SMILES: COC(=O)c1[nH]c2c(c1-c1cccc(F)c1F)CCc1ccccc1-2]

Following a procedure analogous to the one provided for compound of example 1 and replacing 3,5-dichlorophenylboronic acid with an appropriate boronic acid

LCMS (ESI): m/z=340.0 (M+H)⁺

### Example 18: Preparation of methyl 3-(3,5-dimethylphenyl)-4,5-dihydro-1H-benzo[g]indole-2-carboxylate

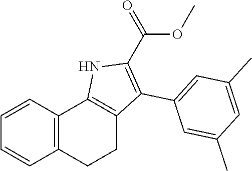 [SMILES: COC(=O)c1[nH]c2c(c1-c1cc(C)cc(C)c1)CCc1ccccc1-2]

Following a procedure analogous to the one provided for compound of example 1 and replacing 3,5-dichlorophenylboronic acid with an appropriate boronic acid

LCMS (ESI): m/z=332.1 (M+H)⁺; ¹H NMR (500 MHz, DMSO) δ=12.04 (s, 1H), 8.03-7.87 (m, 1H), 7.24 (t, J=6.6 Hz, 2H), 7.16-7.10 (m, 1H), 6.98-6.86 (m, 3H), 3.66 (s, 3H), 2.83 (t, J=7.5 Hz, 2H), 2.52 (m, 2H), 2.30 (s, 6H).

### Example 19: Preparation of methyl 3-(2-fluoro-4-methylphenyl)-4,5-dihydro-1H-benzo[g]indole-2-carboxylate

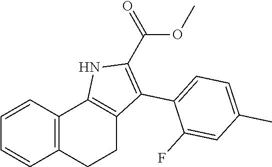 [SMILES: COC(=O)c1[nH]c2c(c1-c1ccc(C)cc1F)CCc1ccccc1-2]

Following a procedure analogous to the one provided for compound of example 1 and replacing 3,5-dichlorophenylboronic acid with an appropriate boronic acid

LCMS (ESI): m/z=336.1 (M+H)⁺; ¹H NMR (500 MHz, DMSO) δ=12.22 (s, 1H), 8.01-7.88 (m, 1H), 7.27-7.19 (m, 3H), 7.18-7.13 (m, 1H), 7.04 (t, J=10.2 Hz, 2H), 3.65 (s, 3H), 2.84 (t, J=7.5 Hz, 2H), 2.63-2.47 (m, 2H), 2.40 (s, 3H).

### Example 20: Preparation of methyl 3-(2,3-dichlorophenyl)-4,5-dihydro-1H-benzo[g]indole-2-carboxylate

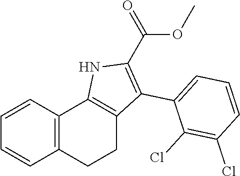 [SMILES: COC(=O)c1[nH]c2c(c1-c1cccc(Cl)c1Cl)CCc1ccccc1-2]

Following a procedure analogous to the one provided for compound of example 1 and replacing 3,5-dichlorophenylboronic acid with an appropriate boronic acid

LCMS (ESI): m/z=372.0 (M+H)⁺, 374.0 (M+2H)⁺; ¹H NMR (500 MHz, DMSO) δ=12.32 (s, 1H), 7.96 (d, J=7.6 Hz, 1H), 7.62 (dd, J=7.9, 1.6 Hz, 1H), 7.38 (t, J=7.8 Hz, 1H), 7.33 (dd, J=7.6, 1.6 Hz, 1H), 7.25 (d, J=7.4 Hz, 2H), 7.20-7.14 (m, 1H), 3.63 (s, 3H), 2.85 (t, J=7.5 Hz, 2H), 2.39 (t, J=7.5, 4.5 Hz, 2H).

### Example 21: Preparation of methyl 3-(pyridin-4-yl)-4,5-dihydro-1H-benzo[g]indole-2-carboxylate

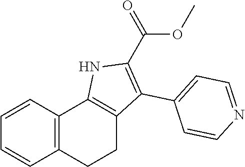 [SMILES: COC(=O)c1[nH]c2c(c1-c1ccncc1)CCc1ccccc1-2]

Following a procedure analogous to the one provided for compound of example 1 and replacing 3,5-dichlorophenylboronic acid with an appropriate boronic acid

LCMS (ESI): m/z=305.1 (M+H)⁺; ¹H NMR (500 MHz, MeOD) δ=8.53-8.39 (m, 2H), 7.65-7.59 (m, 1H), 7.44-7.35 (m, 2H), 7.21-7.12 (m, 2H), 7.12-7.05 (m, 1H), 3.67 (s, 3H), 2.95-2.70 (m, 2H), 2.70-2.39 (m, 2H).

### Example 22: Preparation of methyl 3-(3,5-difluorophenyl)-4,5-dihydro-1H-benzo[g]indole-2-carboxylate

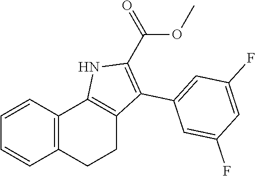 [SMILES: COC(=O)c1[nH]c2c(c1-c1cc(F)cc(F)c1)CCc1ccccc1-2]

Following a procedure analogous to the one provided for compound of example 1 and replacing 3,5-dichlorophenylboronic acid with an appropriate boronic acid.
LCMS (ESI) m/z: 306.0 [M+H]⁺; ¹H NMR (500 MHz, DMSO) δ=12.45 (bs, 1H), 9.13 (s, 1H), 8.83 (s, 2H), 7.99 (d, J=7.0 Hz, 1H), 7.27 (t, J=6.9 Hz, 2H), 7.20-7.17 (m, 1H), 3.71 (s, 3H), 2.88 (t, J=7.5, 2H), 2.59-2.62 (m, 2H).

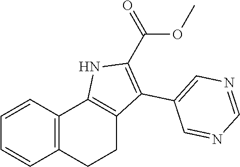 [SMILES: COC(=O)c1[nH]c2c(c1-c1cncnc1)CCc1ccccc1-2]

### Example 24: Preparation of methyl 3-(5-fluoropyridin-3-yl)-4,5-dihydro-1H-benzo[g]indole-2-carboxylate

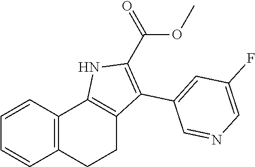 [SMILES: COC(=O)c1[nH]c2c(c1-c1cncc(F)c1)CCc1ccccc1-2]

Following a procedure analogous to the one provided for compound of example 1 and replacing 3,5-dichlorophenylboronic acid with an appropriate boronic acid.

LCMS (ESI) m/z: 323.0 [M+H]⁺; ¹H NMR (500 MHz, DMSO) δ=12.35 (bs, 1H), 8.52 (d, J=2.8 Hz, 1H), 8.45 (t, J=1.7 Hz, 1H), 7.98 (m, 1H), 7.77 (m, 1H), 7.26 (t, J=6.6 Hz, 2H), 7.21-7.15 (m, 1H), 3.71 (s, 3H), 2.87 (t, J=7.5 Hz, 2H), 2.59 (t, J=7.5 Hz, 2H).

### Example 25: Preparation of methyl 3-(5-chloropyridin-3-yl)-4,5-dihydro-1H-benzo[g]indole-2-carboxylate

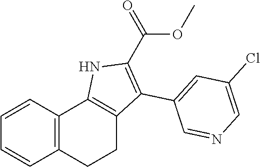 [SMILES: COC(=O)c1[nH]c2c(c1-c1cncc(Cl)c1)CCc1ccccc1-2]

Following a procedure analogous to the one provided for compound of example 1 and replacing 3,5-dichlorophenylboronic acid with an appropriate boronic acid

LCMS (ESI) m/z: 339.0 [M+H]⁺; ¹H NMR (500 MHz, DMSO) δ=12.38 (s, 1H), 8.57 (d, J=2.4 Hz, 1H), 8.53 (d, J=1.8 Hz, 1H), 8.00-7.97 (m, 1H), 7.97-7.94 (m, 1H), 7.28-7.24 (m, 2H), 7.20-7.16 (m, 1H), 3.71 (s, 3H), 2.87 (t, J=7.5 Hz, 2H), 2.62-2.67 (m, 2H).

### Example 26: Preparation of methyl 3-(4-fluoro-3-methoxyphenyl)-4,5-dihydro-1H-benzo[g]indole-2-carboxylate

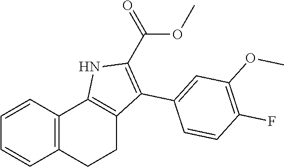 [SMILES: COC(=O)c1[nH]c2c(c1-c1ccc(F)c(OC)c1)CCc1ccccc1-2]

Following a procedure analogous to the one provided for compound of example 1 and replacing 3,5-dichlorophenylboronic acid with an appropriate boronic acid

LCMS (ESI) m/z: 350.1 [M−H]⁻;

### Example 27: Preparation of methyl 3-(3-chloro-5-methylphenyl)-4,5-dihydro-1H-benzo[g]indole-2-carboxylate

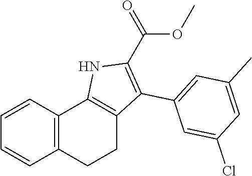 [SMILES: COC(=O)c1[nH]c2c(c1-c1cc(C)cc(Cl)c1)CCc1ccccc1-2]

Following a procedure analogous to the one provided for compound of example 1 and replacing 3,5-dichlorophenylboronic acid with an appropriate boronic acid

LCMS (ESI) m/z: 353.1 [M−H]⁻;

### Example 28: Preparation of methyl 3-(3-chloro-5-fluorophenyl)-4,5-dihydro-1H-benzo[g]indole-2-carboxylate

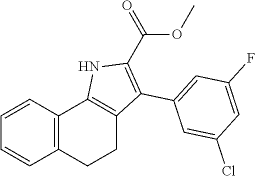 [SMILES: COC(=O)c1[nH]c2c(c1-c1cc(F)cc(Cl)c1)CCc1ccccc1-2]

Following a procedure analogous to the one provided for compound of example 1 and replacing 3,5-dichlorophenylboronic acid with an appropriate boronic acid

LCMS (ESI) m/z: 354.1 [M−H]⁻; ¹H NMR (500 MHz, CDCl₃) δ=9.31 (bs, 1H), 7.39 (d, J=7.6 Hz, 1H), 7.30-7.26 (m, 2H), 7.23-7.18 (m, 2H), 7.09-7.01 (m, 2H), 3.79 (s, 3H), 2.94 (t, J=7.5 Hz, 2H), 2.71-2.63 (m, 2H).

### Example 29: Preparation of methyl 3-(3-chloro-5-methoxyphenyl)-4,5-dihydro-1H-benzo[g]indole-2-carboxylate

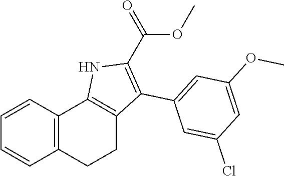 [SMILES: COC(=O)c1[nH]c2c(c1-c1cc(Cl)cc(OC)c1)CCc1ccccc1-2]

Following a procedure analogous to the one provided for compound of example 1 and replacing 3,5-dichlorophenylboronic acid with an appropriate boronic acid

LCMS (ESI) m/z: 368.0 [M+H]⁺; ¹H NMR (500 MHz, CDCl₃) δ=9.29 (bs, 1H), 7.39 (d, J=7.4 Hz, 1H), 7.29-7.27 (m, 1H), 7.25-7.23 (m, 1H), 7.22-7.17 (m, 1H), 7.00 (t, J=1.5 Hz, 1H), 6.88 (t, J=2.1 Hz, 1H), 6.86-6.84 (m, 1H), 3.83 (s, 3H), 3.78 (s, 3H), 2.93 (t, J=7.5 Hz, 2H), 2.71-2.65 (m, 2H).

### Example 30: Preparation of methyl 3-(3,4,5-trichlorophenyl)-4,5-dihydro-1H-benzo[g]indole-2-carboxylate

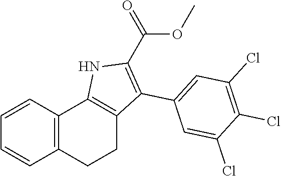 [SMILES: COC(=O)c1[nH]c2c(c1-c1cc(Cl)c(Cl)c(Cl)c1)CCc1ccccc1-2]

Following a procedure analogous to the one provided for compound of example 1 and replacing 3,5-dichlorophenylboronic acid with an appropriate boronic acid LCMS (ESI) m/z: 405.9 [M-H]; ¹H NMR (500 MHZ, CDCl₃) δ=9.32 (bs, 1H), 7.44 (s, 2H), 7.39 (d, J=7.5Hz, 1H), 7.32-7.27 (m, 2H), 7.23-7.20 (m, 1H), 3.80 (s, 3H), 2.94 (t, J=7.5 Hz, 2H), 2.69-2.63 (m, 2H).

### Example 31: Preparation of methyl 3-(3,5-dimethoxyphenyl)-4,5-dihydro-1H-benzo[g]indole-2-carboxylate

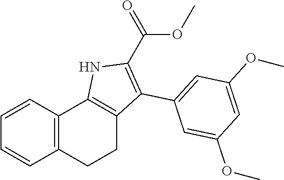 [SMILES: COC(=O)c1[nH]c2c(c1-c1cc(OC)cc(OC)c1)CCc1ccccc1-2]

Following a procedure analogous to the one provided for compound of example 1 and replacing 3,5-dichlorophenylboronic acid with an appropriate boronic acid

LCMS (ESI) m/z: 364.1[M+H]⁺; ¹H NMR (500 MHz, CDCl₃) δ=9.29 (s, 1H), 7.39 (d, J=7.4 Hz, 1H), 7.28 (s, 1H), 7.24 (d, J=7.0 Hz, 1H), 7.21-7.16 (m, 1H), 6.58 (d, J=2.3 Hz, 2H), 6.46 (t, J=2.3 Hz, 1H), 3.82 (s, 6H), 3.78 (s, 3H), 2.92 (t, J=7.5 Hz, 2H), 2.71 (t, J=7.5 Hz, 2H).

### Example 32: Preparation of methyl 3-(3-fluoro-5-methoxyphenyl)-4,5-dihydro-1H-benzo[g]indole-2-carboxylate

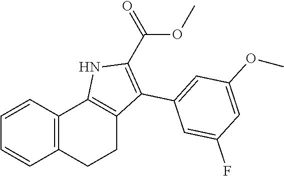 [SMILES: COC(=O)c1[nH]c2c(c1-c1cc(F)cc(OC)c1)CCc1ccccc1-2]

Following a procedure analogous to the one provided for compound of example 1 and replacing 3,5-dichlorophenylboronic acid with an appropriate boronic acid

LCMS (ESI) m/z: 350.1 [M−H]

### Example 33: Preparation of methyl 3-(2,3-dichlorophenyl)-4,5-dihydro-1H-benzo[g]indole-2-carboxylate

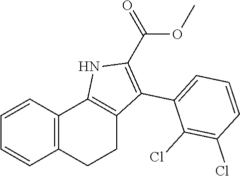 [SMILES: COC(=O)c1[nH]c2c(c1-c1cccc(Cl)c1Cl)CCc1ccccc1-2]

Following a procedure analogous to the one provided for compound of example 1 and replacing 3,5-dichlorophenylboronic acid with an appropriate boronic acid

LCMS (ESI) m/z: 374.0 [M+2H]⁺; ¹H NMR (500 MHz, DMSO) δ=12.32 (bs, 1H), 7.96 (d, J=7.6 Hz, 1H), 7.62 (dd, J=7.9, 1.6 Hz, 1H), 7.38 (t, J=7.8 Hz, 1H), 7.34-7.30 (m, 1H), 7.25 (d, J=7.4 Hz, 2H), 7.20-7.14 (m, 1H), 3.63 (s, 3H), 2.85 (t, J=7.5 Hz, 2H), 2.43-2.36 (m, 2H).

The below Examples-34 to 66 were prepared by following the similar procedure as described in Example-2

### Example 34: 3-(5-Fluoro-2-methoxyphenyl)-4,5-dihydro-1H-benzo[g]indole-2-carboxylic Acid

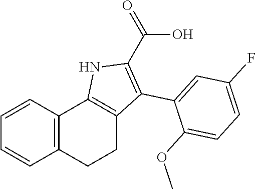 [SMILES: COc1ccc(F)cc1-c1c(C(=O)O)[nH]c2c1CCc1ccccc1-2]

LCMS (ESI): m/z=338.1 (M+H)⁺; ¹H NMR (300 MHz, CHLOROFORM-D) δ=9.34 (bs, 1H), 7.48-7.29 (m, 2H), 7.23 (s, 2H), 7.09-6.95 (m, 2H), 6.95-6.80 (m, 1H), 3.77 (s, 3H), 2.98-2.89 (m, 2H), 2.69-2.43 (m, 2H).

### Example 35: 3-(2-methoxyphenyl)-4,5-dihydro-1H-benzo[g]indole-2-carboxylic Acid

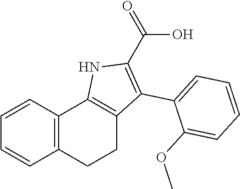 [SMILES: COc1ccccc1-c1c(C(=O)O)[nH]c2c1CCc1ccccc1-2]

LCMS (ESI): m/z=320.1 (M+H)⁺; ¹H NMR (300 MHz, CHLOROFORM-D) δ=9.37 (bs, 1H), 7.42-6.96 (m, 8H), 3.80 (s, 3H), 2.91 (t, J=7.5 Hz, 2H), 2.61-2.52 (m, 2H).

### Example 36: 3-(4-Methoxyphenyl)-4,5-dihydro-1H-benzo[g]indole-2-carboxylic Acid

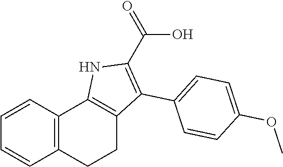 [SMILES: COc1ccc(-c2c(C(=O)O)[nH]c3c2CCc2ccccc2-3)cc1]

LCMS (ESI): m/z=320.1 (M+H)⁺; ¹H NMR (300 MHz, CHLOROFORM-D) δ=9.73 (bs, 1H), 7.67-6.73 (m, 8H), 3.81 (s, 3H), 2.92-2.84 (m, 2H), 2.70-2.64 (m, 2H)

### Example 37: 3-(3,4-Difluorophenyl)-4,5-dihydro-1H-benzo[g]indole-2-carboxylic Acid

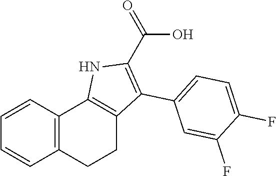 [SMILES: O=C(O)c1[nH]c2c(c1-c1ccc(F)c(F)c1)CCc1ccccc1-2]

LCMS (ESI): m/z=326.0 (M+H)⁺; ¹H NMR (300 MHz, CHLOROFORM-D) δ=9.49 (bs, 1H), 7.40 (d, J=7.2 Hz, 1H), 7.35-7.01 (m, 3H), 7.09-6.87 (m, 2H), 6.79-6.76 (m, 1H), 2.96 (t, J=7.8 Hz, 2H), 2.69 (t, J=7.8 Hz, 2H).

### Example 38: 3-(5-Chloro-2-methoxyphenyl)-4,5-dihydro-1H-benzo[g]indole-2-carboxylic Acid

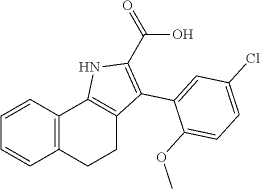 [SMILES: COc1ccc(Cl)cc1-c1c(C(=O)O)[nH]c2c1CCc1ccccc1-2]

LCMS (ESI−): m/z=352.0 (M−H)⁻; ¹H NMR (300 MHz, DMSO-D₆) δ=12.03 (bs, 1H), 12.01 (bs, 1H), 7.90 (d, J=7.9 Hz, 1H), 7.29-7.23 (m, 2H), 7.16 (s, 1H), 7.14-7.05 (m, 2H), 7.04-6.98 (m, 1H), 3.67 (s, 3H), 2.78 (t, J=7.4 Hz, 2H), 2.37 (t, J=7.5 Hz, 2H).

### Example 39: 3-(4-(tert-Butyl)phenyl)-4,5-dihydro-1H-benzo[g]indole-2-carboxylic Acid

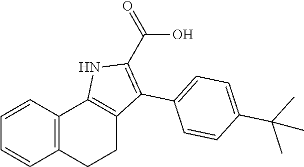 [SMILES: CC(C)(C)c1ccc(-c2c(C(=O)O)[nH]c3c2CCc2ccccc2-3)cc1]

LCMS (ESI): m/z=346.1 (M+H)⁺; ¹H NMR (300 MHz, CHLOROFORM-D) δ=9.42 (s, 1H), 7.41 (m, 4H), 7.23 (dd, J=12.3, 7.1 Hz, 4H), 2.92 (t, J=7.3 Hz, 2H), 2.71 (t, J=7.3 Hz, 2H), 1.39 (s, 9H).

### Example 40: 3-(4-Fluorophenyl)-4,5-dihydro-1H-benzo[g]indole-2-carboxylic acid

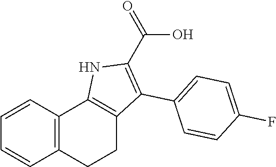 [SMILES: O=C(O)c1[nH]c2c(c1-c1ccc(F)cc1)CCc1ccccc1-2]

LCMS (ESI): m/z=308.0 (M+H)⁺

### Example 41: 3-(2-Fluorophenyl)-4,5-dihydro-1H-benzo[g]indole-2-carboxylic Acid

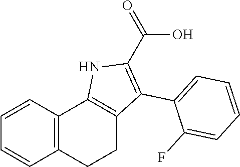 [SMILES: O=C(O)c1[nH]c2c(c1-c1ccccc1F)CCc1ccccc1-2]

LCMS (ESI): m/z=308.1 (M+H)⁺; ¹H NMR (500 MHz, MeOD) δ=7.60-7.55 (m, 1H), 7.25-7.17 (m, 2H), 7.15-7.07 (m, 2H), 7.07-6.95 (m, 3H), 2.76 (t, J=7.5 Hz, 2H), 2.41 (t, J=7.5 Hz, 2H).

### Example 42: 3-(3-Fluorophenyl)-4,5-dihydro-1H-benzo[g]indole-2-carboxylic Acid

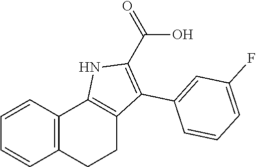 [SMILES: O=C(O)c1[nH]c2c(c1-c1cccc(F)c1)CCc1ccccc1-2]

LCMS (ESI): m/z=308.1 (M+H)⁺; ¹H NMR (500 MHz, MeOD) δ=7.61-7.56 (m, 1H), 7.24 (m, 1H), 7.16-7.08 (m, 2H), 7.08-6.99 (m, 3H), 6.93-6.86 (m, 1H), 2.89-2.67 (m, 2H), 2.49 (m, 2H).

### Example 43: 3-(4-chlorophenyl)-4,5-dihydro-1H-benzo[g]indole-2-carboxylic Acid

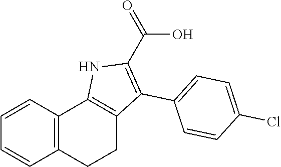 [SMILES: O=C(O)c1[nH]c2c(c1-c1ccc(Cl)cc1)CCc1ccccc1-2]

LCMS (ESI): m/z=322.0 (M+H)⁺; ¹H NMR (500 MHz, DMSO) δ=12.23 (bs, 1H), 8.03-7.95 (m, 1H), 7.45-7.35 (m, 4H), 7.27-7.20 (m, 2H), 7.18-7.12 (m, 1H), 2.77-2.89 (m, 2H), 2.63-2.41 (m, 2H).

### Example 44: 3-(2-chlorophenyl)-4,5-dihydro-1H-benzo[g]indole-2-carboxylic Acid

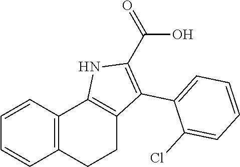 [SMILES: O=C(O)c1[nH]c2c(c1-c1ccccc1Cl)CCc1ccccc1-2]

LCMS (ESI): m/z=324.1 (M+H)⁺; ¹H NMR (500 MHz, MeOD) δ=7.56 (d, J=7.5 Hz, 1H), 7.33-7.29 (m, 1H), 7.20-7.09 (m, 4H), 7.07 (d, J=7.2 Hz, 1H), 7.00 (m, 1H), 2.81-2.67 (m, 2H), 2.40-2.20 (m, 2H).

### Example 45: 3-(3-chlorophenyl)-4,5-dihydro-1H-benzo[g]indole-2-carboxylic Acid

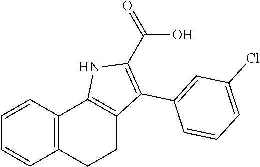 [SMILES: O=C(O)c1[nH]c2c(c1-c1cccc(Cl)c1)CCc1ccccc1-2]

LCMS (ESI): m/z=324.1 (M+H)⁺; ¹H NMR (500 MHz, MeODz) δ=7.58 (d, J=7.5 Hz, 1H), 7.27 (dd, J=5.9, 4.2 Hz, 1H), 7.24-7.19 (m, 1H), 7.19-7.08 (m, 4H), 7.03 (dd, J=7.4, 1.1 Hz, 1H), 2.77 (m, 2H), 2.47 (m, 2H).

### Example 46: 3-(2,4-dichlorophenyl)-4,5-1H-benzo[g]indole-2-carboxylic Acid

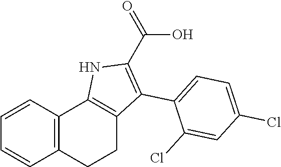 [SMILES: O=C(O)c1[nH]c2c(c1-c1ccc(Cl)cc1Cl)CCc1ccccc1-2]

LCMS (ESI): m/z=360.0 (M+H)⁺; ¹H NMR (500 MHz, MeOD) δ=7.58 (d, J=7.6 Hz, 1H), 7.43-7.36 (m, 1H), 7.24-7.18 (m, 2H), 7.13 (dd, J=15.5, 7.6 Hz, 2H), 7.04 (dd, J=7.4, 1.2 Hz, 1H), 2.78 (t, J=7.6 Hz, 2H), 2.78 (t, J=7.6 Hz, 2H).

### Example 47: 3-(2,3-difluorophenyl)-4,5-dihydro-1H-benzo[g]indole-2-carboxylic Acid

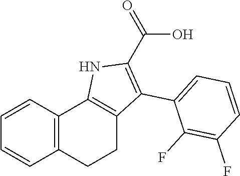 [SMILES: O=C(O)c1[nH]c2c(c1-c1cccc(F)c1F)CCc1ccccc1-2]

LCMS (ESI): m/z=326.1 (M+H)⁺; ¹H NMR (500 MHz, MeOD) δ=7.59 (d, J=7.6 Hz, 1H), 7.23-6.94 (m, 6H), 2.81 (m, 2H), 2.65-2.31 (m, 2H).

### Example 48: 3-(3,5-dimethylphenyl)-4,5-dihydro-1H-benzo[g]indole-2-carboxylic Acid

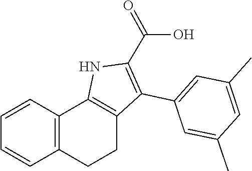 [SMILES: Cc1cc(C)cc(-c2c(C(=O)O)[nH]c3c2CCc2ccccc2-3)c1]

LCMS (ESI): m/z=318.1 (M+H)⁺; ¹H NMR (500 MHz, MeOD) δ=7.57 (d, J=7.1 Hz, 1H), 7.18-7.07 (m, 2H), 7.07-6.97 (m, 1H), 6.88 (d, J=18.7 Hz, 2H), 6.80 (s, 1H), 2.82-2.68 (m, 2H), 2.51-2.41 (m, 2H), 2.23 (s, 6H).

### Example 49: 3-(2-fluoro-4-methylphenyl)-4,5-dihydro-1H-benzo[g]indole-2-carboxylic Acid

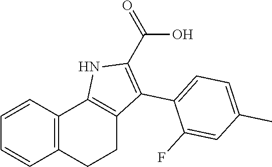 [SMILES: Cc1ccc(-c2c(C(=O)O)[nH]c3c2CCc2ccccc2-3)c(F)c1]

LCMS (ESI): m/z=322.1 (M+H)⁺; ¹H NMR (500 MHz, MeOD) δ=7.56 (d, J=7.6 Hz, 1H), 7.10 (dd, J=19.7, 7.7 Hz, 3H), 7.02 (ddd, J=15.1, 12.1, 11.0 Hz, 1H), 6.87 (m, 2H), 2.75 (t, J=7.5 Hz, 2H), 2.45-2.35 (m, 2H), 2.25 (s, 3H).

### Example 50: 3-(2,3-dichlorophenyl)-4,5-dihydro-1H-benzo[g]indole-2-carboxylic Acid

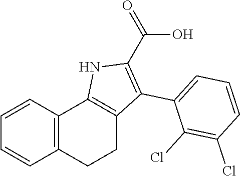 [SMILES: O=C(O)c1[nH]c2c(c1-c1cccc(Cl)c1Cl)CCc1ccccc1-2]

LCMS (ESI) m/z: 360.0 [M+2H]⁺; ¹H NMR (500 MHz, MeOD) δ=7.58 (d, J=7.6 Hz, 1H), 7.41-7.35 (m, 1H), 7.18-7.15 (m, 2H), 7.14-7.09 (m, 2H), 7.04 (dd, J=7.4, 1.2 Hz, 1H), 2.82-2.73 (m, 2H), 2.38-2.28 (m, 2H).

### Example 51: 3-(pyridin-4-yl)-4,5-dihydro-1H-benzo[g]indole-2-carboxylic Acid

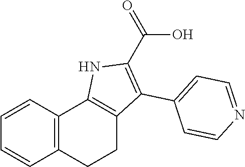 [SMILES: O=C(O)c1[nH]c2c(c1-c1ccncc1)CCc1ccccc1-2]

LCMS (ESI): m/z=291.1 (M+H)⁺; ¹H NMR (500 MHz, MeOD) δ=8.41 (d, J=6.0 Hz, 2H), 7.59 (d, J=7.7 Hz, 1H), 7.42-7.37 (m, 2H), 7.14 (d, J=9.3 Hz, 2H), 7.07 (d, J=7.4 Hz, 1H), 2.81 (t, J=7.5 Hz, 2H), 2.59-2.50 (m, 2H).

### Example 52: 3-(3,5-difluorophenyl)-4,5-dihydro-1H-benzo[g]indole-2-carboxylic Acid

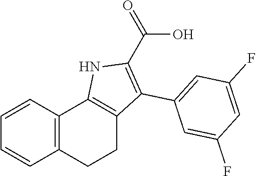 [SMILES: O=C(O)c1[nH]c2c(c1-c1cc(F)cc(F)c1)CCc1ccccc1-2]

LCMS (ESI) m/z: 324.0 [M−2H]⁻; ¹H NMR (500 MHz, MeOD) δ=7.59 (d, J=7.7 Hz, 2H), 7.17-7.11 (m, 2H), 7.07-7.04 (m, 1H), 6.88-6.86 (m, 2H), 6.75 (m, 1H), 2.80 (t, J=7.5 Hz, 1H), 2.55-2.50 (m, 1H).

### Example 53: 3-(5-fluoropyridin-3-yl)-4,5-dihydro-1H-benzo[g]indole-2-carboxylic Acid

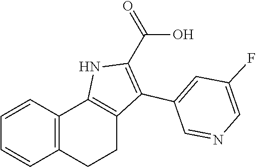 [SMILES: O=C(O)c1[nH]c2c(c1-c1cncc(F)c1)CCc1ccccc1-2]

LCMS (ESI) m/z: 309.0 [M+H]⁺; ¹H NMR (500 MHz, MeOD) δ=8.32 (s, 1H), 8.27 (d, J=2.6 Hz, 1H), 7.63-7.58 (m, 2H), 7.17-7.13 (m, 2H), 7.09-7.04 (m, 1H), 2.82 (t, J=7.5 Hz, 1H), 2.56-2.53 (m, 1H).

### Example 54: 3-(5-chloropyridin-3-yl)-4,5-dihydro-1H-benzo[g]indole-2-carboxylic Acid

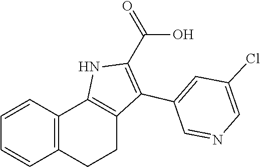 [SMILES: O=C(O)c1[nH]c2c(c1-c1cncc(Cl)c1)CCc1ccccc1-2]

LCMS (ESI) m/z: 325.0 [M+H]⁺; ¹H NMR (500 MHz, DMSO) δ=12.44 (s, 1H), 12.26 (s, 1H), 8.54 (dd, J=9.0, 1.7 Hz, 2H), 7.98 (d, J=7.6 Hz, 1H), 7.95-7.92 (m, 1H), 7.24 (t, J=7.5 Hz, 2H), 7.16 (t, J=7.3 Hz, 1H), 2.86 (t, J=7.4 Hz, 2H), 2.58 (t, J=7.5 Hz, 2H).

### Example 55: 3-(pyrimidin-5-yl)-4,5-dihydro-1H-benzo[g]indole-2-carboxylic Acid

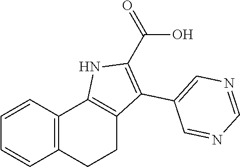 [SMILES: O=C(O)c1[nH]c2c(c1-c1cncnc1)CCc1ccccc1-2]

LCMS (ESI) m/z: 292.3 [M+H]⁺

### Example 56: 3-(4-fluoro-2-methoxyphenyl)-4,5-dihydro-1H-benzo[g]indole-2-carboxylic Acid

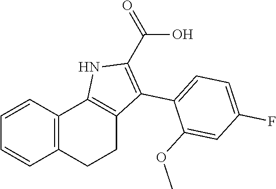 [SMILES: COc1cc(F)ccc1-c1c(C(=O)O)[nH]c2c1CCc1ccccc1-2]

LCMS (ESI) m/z: 336.1 [M−H]⁻; ¹H NMR (500 MHz, MeOD) δ=7.55 (d, J=7.5 Hz, 1H), 7.15-7.04 (m, 3H), 7.02 (t, J=7.0 Hz, 1H), 6.68 (dd, J=11.2, 2.3 Hz, 1H), 6.59-6.54 (m, 1H), 3.69-3.61 (s, 3H), 2.75 (t, J=7.5 Hz, 2H), 2.35 (t, J=7.5 Hz, 2H).

### Example 57: 3-(3-chloro-5-methylphenyl)-4,5-dihydro-1H-benzo[g]indole-2-carboxylic Acid

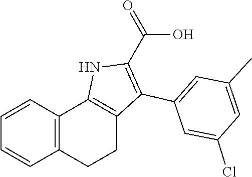 [SMILES: Cc1cc(Cl)cc(-c2c(C(=O)O)[nH]c3c2CCc2ccccc2-3)c1]

LCMS (ESI) m/z: 337.8, 339.5 [M+H]⁺; ¹H NMR (500 MHz, MeOD) δ=7.70 (d, J=7.1 Hz, 1H), 7.27-7.21 (m, 2H), 7.18 (s, 1H), 7.17-7.14 (m, 1H), 7.13-7.10 (m, 2H), 2.89 (t, J=7.5 Hz, 2H), 2.63-2.55 (m, 2H), 2.37 (s, 3H).

### Example 58: 3-(3-chloro-5-fluorophenyl)-4,5-dihydro-1H-benzo[g]indole-2-carboxylic Acid

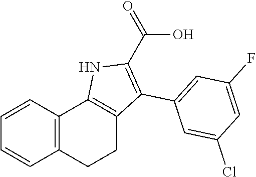 [SMILES: O=C(O)c1[nH]c2c(c1-c1cc(F)cc(Cl)c1)CCc1ccccc1-2]

LCMS (ESI) m/z: 340.0 [M−H]⁻; ¹H NMR (500 MHz, MeOD) δ=7.58 (d, J=7.6 Hz, 1H), 7.17-7.09 (m, 3H), 7.07-7.02 (m, 1H), 7.02-6.94 (m, 2H), 2.78 (t, J=7.5 Hz, 2H), 2.53-2.44 (m, 2H).

### Example 59: 3-(3-chloro-5-methoxyphenyl)-4,5-dihydro-1H-benzo[g]indole-2-carboxylic Acid

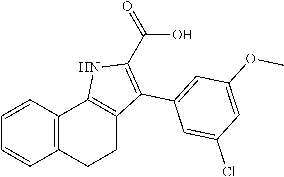 [SMILES: COc1cc(Cl)cc(-c2c(C(=O)O)[nH]c3c2CCc2ccccc2-3)c1]

LCMS (ESI) m/z: 352.0 [M−H]⁻; ¹H NMR (500 MHz, MeOD) δ=7.57 (d, J=7.5 Hz, 1H), 7.16-7.07 (m, 2H), 7.05-7.00 (m, 1H), 6.86-6.82 (m, 1H), 6.79-6.72 (m, 2H), 3.69 (s, 3H), 2.77 (t, J=7.5 Hz, 2H), 2.52-2.43 (m, 2H).

### Example 60: 3-(3,4,5-trichlorophenyl)-4,5-dihydro-1H-benzo[g]indole-2-carboxylic Acid

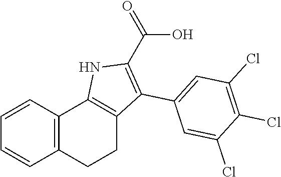 [SMILES: O=C(O)c1[nH]c2c(c1-c1cc(Cl)c(Cl)c(Cl)c1)CCc1ccccc1-2]

LCMS (ESI) m/z: 390.0, 391.9 [M−H]⁻; ¹H NMR (500 MHz, MeOD) δ=7.59 (d, J=7.6 Hz, 1H), 7.41 (s, 2H), 7.17-7.11 (m, 2H), 7.09-7.04 (m, 1H), 2.81 (t, J=7.5 Hz, 2H), 2.52 (t, J=7.5 Hz, 2H).

### Example 61: 3-(3,5-dimethoxyphenyl)-4,5-dihydro-1H-benzo[g]indole-2-carboxylic Acid

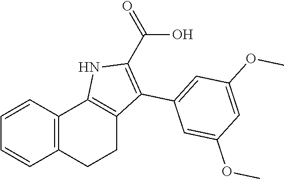 [SMILES: COc1cc(OC)cc(-c2c(C(=O)O)[nH]c3c2CCc2ccccc2-3)c1]

LCMS (ESI) m/z: 348.1 [M−H]⁻; ¹H NMR (500 MHz, DMSO) δ=11.98 (s, 1H), 7.96 (s, 1H), 7.35-7.05 (m, 3H), 6.60-6.35 (m, 3H), 3.75 (s, 6H), 2.8-2.7 (m, 2H), 2.65-2.55 (m, 2H).

### Example 62: 3-(3-fluoro-5-methoxyphenyl)-4,5-dihydro-1H-benzo[g]indole-2-carboxylic Acid

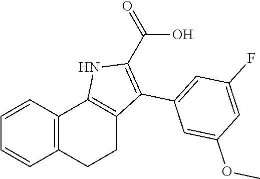 [SMILES: COc1cc(F)cc(-c2c(C(=O)O)[nH]c3c2CCc2ccccc2-3)c1]

LCMS (ESI) m/z: 336.1 [M−H]⁻; ¹H NMR (500 MHz, MeOD) δ=7.58 (d, J=7.6 Hz, 1H), 7.15-7.08 (m, 2H), 7.05-7.01 (m, 1H), 6.66-6.63 (m, 1H), 6.61-6.56 (m, 1H), 6.52-6.48 (m, 1H), 3.70 (s, 3H), 2.77 (t, J=7.5 Hz, 2H), 2.53-2.46 (m, 2H).

### Example 63: 3-phenyl-4,5-dihydro-1H-benzo[g]indole-2-carboxylic Acid

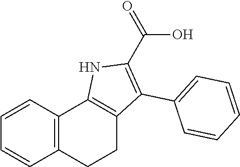 [SMILES: O=C(O)c1[nH]c2c(c1-c1ccccc1)CCc1ccccc1-2]

LCMS (ESI) m/z: 290.2 [M+H]⁺; ¹H NMR (500 MHz, MeOD) δ=7.58 (d, J=7.7 Hz, 1H), 7.30-7.26 (m, 2H), 7.26-7.23 (m, 2H), 7.19-7.165 (m, 1H), 7.16-7.10 (m, 2H), 7.06-7.01 (m, 1H), 2.78 (t, J=7.5 Hz, 2H), 2.53-2.46 (m, 2H).

### Example 64: 3-(3-chloro-5-fluorophenyl)-5-methyl-4,5-dihydro-1H-benzo[g]indole-2-carboxylic Acid

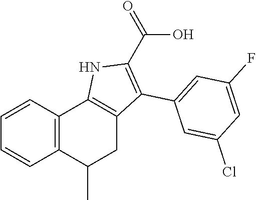 [SMILES: CC1Cc2c([nH]c(C(=O)O)c2-c2cc(F)cc(Cl)c2)-c2ccccc21]

LCMS (ESI) m/z: 356.3 [M+H]⁺; ¹H NMR (500 MHz, MeOD) δ=7.61 (dd, J=7.5, 1.1 Hz, 1H), 7.18-7.12 (m, 2H), 7.11-7.07 (m, 2H), 7.03-6.99 (m, 1H), 6.98-6.95 (m, 1H), 2.97-2.94 (m, 1H), 2.69 (dd, J=15.4, 6.1 Hz, 1H), 2.31 (dd, J=15.4, 5.8 Hz, 1H), 1.09 (d, J=7.0 Hz, 3H).

### Example 65: 7-bromo-3-(3,5-dichlorophenyl)-4,5-dihydro-1H-benzo[g]indole-2-carboxylic Acid

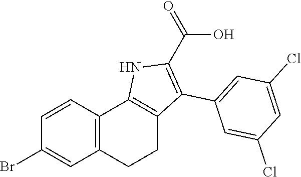 [SMILES: O=C(O)c1[nH]c2c(c1-c1cc(Cl)cc(Cl)c1)CCc1cc(Br)ccc1-2]

LCMS (ESI) m/z: [M+H]⁺ 435.1

### Prophetic Examples

The below list of examples 66 to 75 given in Table-1 can be prepared by following the similar procedure as described in example-1 and then Example-2 by taking Intermediate-1 and appropriately substituted boronic acids/esters.

TABLE 1

| Ex. | | |
|---|---|---|
| No. | Structure | Chemical Name |
| 66 | 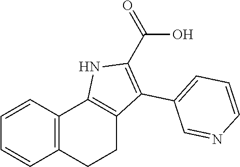 [SMILES: O=C(O)c1[nH]c2c(c1-c1cccnc1)CCc1ccccc1-2] | 3-(pyridin-3-yl)-4,5-dihydro-1H- benzo[g]indole-2-carboxylic acid |
| 67 | 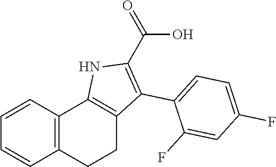 [SMILES: O=C(O)c1[nH]c2c(c1-c1ccc(F)cc1F)CCc1ccccc1-2] | 3-(2,4-difluorophenyl)-4,5-dihydro- 1H-benzo[g]indole-2-carboxylic acid |
| 68 | 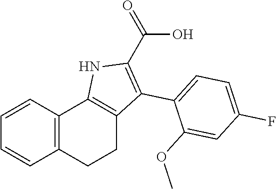 [SMILES: COc1cc(F)ccc1-c1c(C(=O)O)[nH]c2c1CCc1ccccc1-2] | 3-(4-fluoro-2-methoxyphenyl)-4,5- dihydro-1H-benzo[g]indole-2- carboxylic acid |
| 69 | 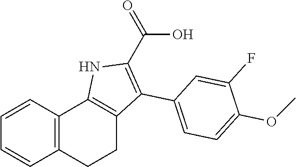 [SMILES: COc1ccc(-c2c(C(=O)O)[nH]c3c2CCc2ccccc2-3)cc1F] | 3-(3-fluoro-4-methoxyphenyl)-4,5- dihydro-1H-benzo[g]indole-2- carboxylic acid |
| 70 | 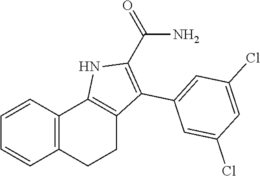 [SMILES: NC(=O)c1[nH]c2c(c1-c1cc(Cl)cc(Cl)c1)CCc1ccccc1-2] | 3-(3,5-dichlorophenyl)-4,5-dihydro- 1H-benzo[g]indole-2-carboxamide |
| 71 | 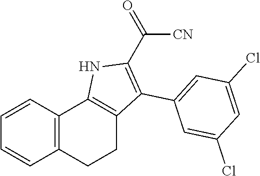 [SMILES: N#CC(=O)c1[nH]c2c(c1-c1cc(Cl)cc(Cl)c1)CCc1ccccc1-2] | 3-(3,5-dichlorophenyl)-4,5-dihydro- 1H-benzo[g]indole-2-carbonyl cyanide |
| 72 | 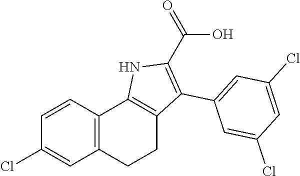 [SMILES: O=C(O)c1[nH]c2c(c1-c1cc(Cl)cc(Cl)c1)CCc1cc(Cl)ccc1-2] | 7-bromo-3-(3,5-dichlorophenyl)-4,5- dihydro-1H-benzo[g]indole-2- carboxylic acid |
| 73 | 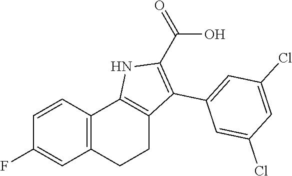 [SMILES: O=C(O)c1[nH]c2c(c1-c1cc(Cl)cc(Cl)c1)CCc1cc(F)ccc1-2] | 3-(3,5-dichlorophenyl)-7-fluoro-4,5- dihydro-1H-benzo[g]indole-2- carboxylic acid |
| 74 | 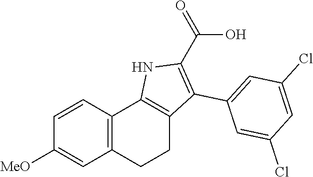 [SMILES: COc1ccc2c(c1)CCc1c-2[nH]c(C(=O)O)c1-c1cc(Cl)cc(Cl)c1] | 3-(3,5-dichlorophenyl)-7-methoxy- 4,5-dihydro-1H-benzo[g]indole-2- carboxylic acid |
| 75 | 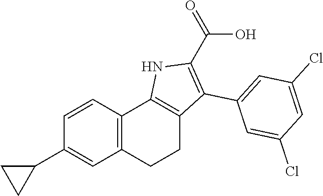 [SMILES: O=C(O)c1[nH]c2c(c1-c1cc(Cl)cc(Cl)c1)CCc1cc(C3CC3)ccc1-2] | 7-cyclopropyl-3-(3,5- dichlorophenyl)-4,5-dihydro-1H- benzo[g]indole-2-carboxylic acid |

### Biological Assays

### Enzyme Activity: Determination of IC₅₀

The Class B beta-lactamases activities were measured in the presence of the test inhibitors in a fluorescence assay against an in-house synthesized fluorescent cephalosporin substrate FC5 (Berkel, et. al. J. Med. Chem. 2013, 56(17), 6945). The enzymes (NDM-1, IMP-1) and the substrate were diluted in 20 mM HEPES, pH 7.4, supplemented with 300 mM NaCl and 10 μM ZnSO₄. In the assay, the final concentration of enzyme was 50 pM, and 100 pM for NDM-1, and IMP-1 respectively, and the final concentration of FC5 was 1.5 μM for NDM-1 and 10 μM for IMP-1. The test inhibitors/compounds were dissolved in dimethylsulfoxide (DMSO) and diluted in the assay with assay buffer (20 mM HEPES, pH 7.4, supplemented with 5% DMSO, 300 mM NaCl and 10 μM ZnSO₄), resulting in a final concentration range of 0.008 μM to 25 μM. The assays were performed in 96-well microplate (flat bottom, black). The test inhibitors were incubated with the MBL enzyme for 10 min at room temperature, followed by addition the substrate and the fluorescence was recorded immediately (λex 380 nm, λem 460 nm) on a microplate reader. Using the initial velocity data plotted against the inhibitor concentration, the half-maximal inhibitory concentrations were calculated by an IC₅₀ curve-fitting model in GraphPad Prism 9 software. Representative compounds of the present invention exhibit inhibition of Class B β-lactamases in this assay. For example, the compounds of examples 2, 34-64 were tested in this assay and were found to have IC₅₀ values shown in Table 1.

### Table 1: IC₅₀ Values of Substituted Tricyclic Heterocyclic Compounds Against NDM-1 and IMP-1

TABLE 1
IC₅₀ values substituted tricyclic heterocyclic
compounds against NDM-1 and IMP-1

| | | IC₅₀ (μM) | IC₅₀ (μM) |
|---|---|---|---|
| S. No | CODE | NDM-1 | IMP-1 |
| 1 | Example 2 | 0.036 | 0.026 |
| 2 | Example 34 | 0.103 | 0.096 |
| 3 | Example 35 | 0.248 | 1.810 |
| 4 | Example 36 | 2.653 | 18.285 |
| 5 | Example 37 | 0.342 | 3.856 |
| 6 | Example 38 | 0.026 | 0.026 |
| 7 | Example 39 | 0.591 | 72.094 |
| 8 | Example 41 | 0.256 | 1.344 |
| 9 | Example 42 | 0.079 | 1.149 |
| 10 | Example 43 | 0.007 | 1.484 |
| 11 | Example 44 | 0.019 | 1.103 |
| 12 | Example 45 | 0.132 | ND |
| 13 | Example 46 | 0.306 | 10.445 |
| 14 | Example 47 | 0.323 | 3.299 |
| 15 | Example 48 | 0.252 | 0.695 |
| 16 | Example 49 | 0.319 | 2.054 |
| 17 | Example 50 | 0.211 | 24.995 |
| 18 | Example 51 | 0.604 | 11.065 |
| 19 | Example 52 | 0.158 | 4.871 |
| 20 | Example 53 | 0.334 | 3.744 |
| 21 | Example 54 | 0.075 | 1.359 |
| 22 | Example 56 | 0.143 | 2.672 |
| 23 | Example 57 | 1.598 | ND |
| 24 | Example 58 | 0.009 | 0.869 |
| 25 | Example 59 | 0.015 | 0.552 |
| 26 | Example 60 | ND | 1.563 |
| 27 | Example 61 | 0.049 | 0.495 |
| 28 | Example 62 | 0.196 | 3.447 |
| 29 | Example 63 | 2.358 | 4.055 |
| 30 | Example 64 | 1.417 | 0.821 |

ND: not determined

### In-Vitro Pharmacological Activity

### Antibiotic Activity of Imipenem and Meropenem on NDM-1 Expressing Bacteria in the Presence of Representative Compounds of the Invention

### Bacterial Strains

The strain included in the study was Klebsiella pneumoniae ATCC BAA-2146 (Himedia®, India) with presence of New Delhi metallo-β-lactamase (NDM-1) gene. The quality control reference strains Escherichia coli ATCC 25922 and Pseudomonas aeruginosa ATCC 27853 were procured from Himedia®, India.

### Minimum Inhibitory Concentration (MIC)

MIC values were identified through broth-microdilution in sterilized 96-well polystyrene flat-bottom microtitre plates (Cole-Parmer®) according to the guideline of the Clinical and Laboratory Standards Institute (CLSI) (CLSI; Methods for dilution antimicrobial susceptibility tests for bacteria that grow aerobically; Clinical Laboratory Standards Institute, M07-A09, 2012). This method involves the use of BBL™ Cation-adjusted Mueller-Hinton II Broth (CA-MHB; BD) and the strain (Klebsiella pneumoniae ATCC BAA-2146) concentration was adjusted to 5×10⁵ CFU/mL. After 16-18 h of incubation at various concentrations of Imipenem, Meropenem (Alfa Aesar), or test compounds at 37° C., MIC was defined as the lowest concentration of antibiotic or test compound with no visible growth.

### Checkerboard Synergy Assay

The fold modulation activity of test compounds, in combination with Imipenem or Meropenem, was checked against Klebsiella pneumoniae ATCC BAA-2146 strain by the broth-microdilution checkerboard synergy assay. The plates contained 5×10⁵ CFU/mL bacterial inocula and 2-fold serial dilutions of the antibiotics (128-2 μg/mL) as well as test compounds (32-4 μg/mL) in a total volume of 200 μL of CA-MHB. Dimethyl sulfoxide (DMSO, ≤2.5%) was included as vehicle control. Following 16-18 h of incubation at 37° C., the MICs of antibiotics as well as drug combinations were visually inspected.

### Table 2: Synergistic Effect of Compounds on MIC of Imipenem and Meropenem Against Strain of Klebsiella Pneumoniae ATCC BAA-2146

| | | | | | | | | Fold reduction in | | | |
|---|---|---|---|---|---|---|---|---|---|---|---|
| | | | Fold reduction in Imipenem | | | | | meropenem MIC in the | | | |
| | | Intrinsic | MIC in the presence of | | | | Intrinsic | presence of compounds | | | |
| | | MIC | compounds (μg/mL) | | | | MIC | (μg/mL) | | | |
| S. No | CODE | (μg/mL) | 32 | 16 | 8 | 4 | (μg/mL) | 32 | 16 | 8 | 4 |
| 1 | Example 2 | >64/>128 | 32/16 | 16/8 | 8/8 | 8/4 | >128 | 16 | 8 | 8 | 4 |
| 2 | Example 34 | >128 | 16 | 8 | 8 | 4 | >128 | 16 | 4 | 4 | 4 |
| 3 | Example 35 | >128 | 8 | 4 | 4 | 2 | >128 | 8 | 8 | 4 | 4 |
| 4 | Example 36 | >64 | 4 | 2 | 2 | 2 | >64 | 4 | 4 | 2 | 2 |
| 5 | Example 37 | >64 | 4 | 4 | 2 | 2 | >64 | 4 | 4 | 4 | 4 |
| 6 | Example 38 | >128 | 8 | 8 | 4 | 4 | >128 | 8 | 4 | 4 | 4 |
| 7 | Example 39 | >64 | 4 | 4 | 4 | 2 | >64 | 4 | 4 | 4 | 4 |
| 8 | Example 41 | >128 | 8 | 8 | 4 | 4 | >128 | 8 | 4 | 4 | 4 |
| 9 | Example 42 | >128 | 8 | 8 | 4 | 4 | >128 | 8 | 4 | 4 | 4 |
| 10 | Example 43 | >128 | 8 | 4 | 4 | 4 | >128 | 4 | 4 | 4 | 4 |
| 11 | Example 44 | >128 | 4 | 4 | 4 | 4 | >128 | 4 | 4 | 4 | 4 |
| 12 | Example 45 | >128 | 8 | 8 | 4 | 2 | >128 | 8 | 4 | 4 | 2 |
| 13 | Example 46 | >128 | 16 | 8 | 4 | 4 | >128 | 16 | 8 | 4 | 4 |
| 14 | Example 47 | >128 | 8 | 4 | 4 | 2 | >128 | 4 | 4 | 4 | 2 |
| 15 | Example 48 | >128 | 8 | 8 | 4 | 4 | >128 | 8 | 4 | 4 | 4 |
| 16 | Example 49 | >128 | ND | ND | ND | ND | >128 | 4 | 4 | 2 | 2 |
| 17 | Example 50 | >128 | 4 | 4 | 2 | 2 | >128 | 4 | 2 | 2 | — |
| 18 | Example 51 | >128 | 8 | 8 | 4 | 4 | >128 | 8 | 8 | 4 | 4 |
| 19 | Example 52 | >128 | 8 | 8 | 4 | 4 | >128 | 4 | 2 | 2 | — |
| 20 | Example 53 | >128 | 8 | 4 | 4 | 4 | >128 | 4 | 4 | 2 | ND |
| 21 | Example 54 | >128 | 8 | 8 | 4 | 4 | >128 | 8 | 8 | 4 | 4 |
| 22 | Example 56 | >128 | 2 | ND | ND | ND | >128 | 8 | 4 | 4 | 4 |
| 23 | Example 57 | >128 | 16 | 8 | 8 | 4 | >128 | 8 | 8 | 4 | 4 |
| 24 | Example 58 | >128 | 16 | 8 | 8 | 4 | >128 | ND | ND | ND | ND |
| 25 | Example 59 | >128 | 16 | 8 | 4 | 4 | >128 | 16 | 8 | 8 | 4 |
| 26 | Example 60 | >128 | 16 | 8 | 8 | 4 | >128 | 8 | 8 | 8 | 4 |
| 27 | Example 61 | >128 | 8 | 8 | 8 | 4 | >128 | 8 | 8 | 4 | 4 |
| 28 | Example 62 | >128 | 8 | 8 | 4 | 4 | >128 | ND | ND | ND | ND |
| 29 | Example 63 | >128 | 8 | 8 | 4 | 4 | >128 | ND | ND | ND | ND |
| 30 | Example 64 | >128 | 8 | 8 | 4 | 4 | >128 | ND | ND | ND | ND |

Note:
The MIC of meropenem against Klebsiella pneumoniae ATCC BAA-2146 is 128 μg/mL.

Thus, the above in-vitro assays method shows that the compounds of the invention were found to have inhibition against MBLs (NDM-1 and IMP-1) in biochemical assay and shown synergistic effect in combination with beta-lactam antibiotics in carbapenem resistance strain, thereby showing utility for treating diseases, disorders associated with the modulation of MBL and antibiotic resistance.

The compound testing results have demonstrated that the substituted tricyclic compounds of formula (I) are capable of inhibiting clinically important MBLs. The substituted tricyclic-based scaffold described in this invention can be developed into broad-spectrum high-affinity inhibitors targeting multiple beta-lactamases in resistant bacteria, and can be combined with beta-lactam antibiotics to treat infections caused by multi-resistant bacteria.

Through the use of above described assay method, compounds were found to exhibit fold modulation activity with antibiotic, thus to be particularly well suited for the treatment of the diseases or disorders as described herein above.

### Advantages of the Invention

As we have reached a point where for the patients infected with multi-drug resistant bacteria, there is no magic pellet. The present innovation is about small molecules that inhibits MBL enzyme and fold modulation of last resort carbapenems (β-lactam antibiotics) class antibiotics. These inhibitors can help in retaining the antibiotic activity in case of resistance and therefore can be beneficial to across the globe as huge unmet medical need is present.

We claim:

1. A compound of formula I, 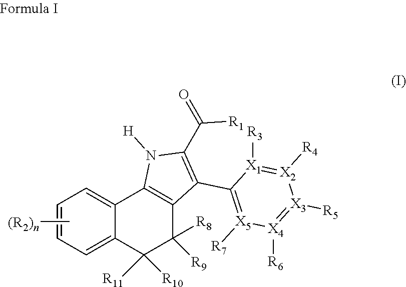
a stereoisomer thereof, or a pharmaceutically acceptable salt thereof
wherein
R₁ is selected from the group consisting of
(i) —OH;
(ii) —NRₐR_{b}, wherein Rₐ and R_{b} are independently selected at each occurrence from hydrogen, unsubstituted or substituted alkyl, and unsubstituted or substituted cycloalkyl; methoxy; or Rₐ and R_{b} together with the carbon atom to which they are attached, may form a substituted or unsubstituted 3 to 7 membered saturated carbocyclic ring;
(iii) —OR_{c}, wherein R_{c} is selected from C₁₋₆ alkyl, C₃₋₈ cycloalkyl, C₃₋₈ cycloalkyl, C₁₋₂ alkyl, aryl, aryl-C₁₋₂ alkyl, heteroaryl, heteroaryl-C₁₋₂ alkyl, heterocyclyl or heterocyclyl-C₁₋₂ alkyl, each of which is optionally substituted by one or more substituent groups;
(iv) —R_{d}, wherein R_{d} is independently selected at each occurrence from hydrogen, substituted or unsubstituted alkyl, and substituted or unsubstituted cycloalkyl;
R₂, which may be same or different at each occurrence, is independently selected from the group consisting of halogen, cyano, nitro, substituted or unsubstituted alkyl, substituted or unsubstituted haloalkyl, substituted or unsubstituted cycloalkyl, —C(O)ORₑ, —OC(O)ORₑ, —O(CRₐR_{b})r-C(O)ORₑ, —(CRₐR_{b})ᵣ—C(O)ORₑ, —C(O)Rₕ, NR_{f}R_{g}, —C(O)NR_{f}R_{g}, —NR_{f}C(O)Rₕ, —NR_{f}S(O)₂R_{g}, —S(O)₀₋₂Rₑ, and —S(O)₂NR_{f}R_{g},
Rₐ and R_{b}, which may be same or different at each occurrence, are independently selected from the group consisting of hydrogen, halogen, hydroxy, cyano, substituted or unsubstituted haloalkyl, substituted or unsubstituted alkyl and substituted or unsubstituted cycloalkyl; or Rₐ and R_{b} together with the carbon atom to which they are attached, may form a unsubstituted or substituted 3 to 7 membered saturated carbocyclic ring;
Rₑ, which may be same or different at each occurrence, is independently selected from the group consisting of hydrogen, substituted or unsubstituted aryl, substituted or unsubstituted haloalkyl, and substituted or unsubstituted alkyl;
R_{f} and R_{g}, which may be same or different at each occurrence, are independently selected from the group consisting of hydrogen, substituted or unsubstituted alkyl, —(CRaRb)ᵣ-C(O)ORₑ, substituted or unsubstituted cycloalkyl, substituted or unsubstituted cycloalkylalkyl, substituted or unsubstituted aryl, substituted or unsubstituted arylalkyl, substituted or unsubstituted heteroaryl, substituted or unsubstituted heteroarylalkyl, substituted or unsubstituted heterocyclyl, and substituted or unsubstituted heterocyclylalkyl; or R_{f} and R_{g} together with the nitrogen atom to which they are attached, may form a substituted or unsubstituted, saturated or unsaturated 3 to 10 membered cyclic ring, wherein the unsaturated cyclic ring may have one or two double bonds; at each occurrence, Rₕ is substituted or unsubstituted alkyl or substituted or unsubstituted aryl;
‘n’ is an integer ranging from 0 to 5, both inclusive;
‘r’ is an integer ranging from 1 to 3, both inclusive;
X₁, X₂, X₃, X₃ or X₅ are selected from C or N, with the proviso that only one or two of X₁, X₂, X₃, X₃ or X₅ can be N;
R₃ is selected from hydrogen, halo, cyano, nitro, hydroxy or a group -A¹-B¹-C¹
wherein, A¹ is absent or a linker group of the formula —[CR^{1A}R^{1B}]ₚ—in which p is an integer selected from 1 or 2, 3 or 4, and R^{1A} and R^{1B} are each independently selected from hydrogen or C₁₋₂ alkyl;
B¹ is absent or —O—, —C(O)—, —C(O)O—, —OC(O)—, —CH(OR^{1C})—, —N(R^{1C})—, N(R^{1D})C(O), —N(R^{1D})—C(O)O—, —C(O)—N(R^{1C})—, —N(R^{1D})C(O)N(R^{1C})—, —S—, —SO—, —SO₂—, —S(O)₂N(R^{1C})—, or —N(R^{1D})SO₂— wherein R^{1C} and R^{1D} are each independently selected from hydrogen or methyl; and
C¹ is hydrogen, C₁₋₆ alkyl, C₂₋₄ alkenyl, C₂₋₄ alkynyl, aryl, C₃₋₄ cycloalkyl, C₃₋₆ cycloalkenyl, heterocyclyl, or heteroaryl;
and wherein C¹ is optionally further substituted by one or more substituent groups independently selected from oxo, halo, cyano, nitro, hydroxy, carboxy, NR^{1E}R^{1F}, C₁₋₄ alkoxy, C₁₋₄ alkyl, C₃₋₈ cycloalkyl, C₃₋₈ cycloalkyl-C₁₋₃ alkyl, C₁₋₄ alkanoyl, C₁₋₄ alkylsulphonyl, aryl, aryloxy, aryl-C₁₋₂ alkyl, heterocyclyl, heterocyclyloxy, heterocyclyl-C₁₋₂ alkyl, heteroaryl, heteroaryloxy, heteroaryl-C₁₋₂ alkyl, C(O)NR^{1E}R^{1F}, NR^{1E}C(O)R^{1F}, NR^{1E}S(O)₂R^{1F} and S(O)₂NR^{1E}R^{1F}; wherein R^{1E} and R^{1F} are each independently selected from hydrogen, C₁₋₄ alkyl or C₃₋₆ cycloalkyl or C₃₋₆ cycloalkyl C₁₋₂ alkyl; or R^{1E} and R^{1F} can be linked such that, together with the nitrogen atom to which they are attached, they form a 4-7 membered heterocyclic ring;
and wherein any alkyl, aryl, heterocyclyl or heteroaryl group present in a substituent group on C¹ is optionally further substituted by halo, cyano, nitro, hydroxy, caboxy, NR^{1G}R^{1H}, C₁₋₂ alkoxy, or C₁₋₂ alkyl; wherein R^{1G} and R^{1H} are selected from hydrogen or C₁₋₂ alkyl;
or R^{1C} and C¹ can be linked such that, together with the nitrogen atom to which they are attached, they form a 4-7 membered heterocyclic ring, which is optionally substituted by oxo, halo, cyano, nitro, hydroxy, carboxy, NR^{1E}R^{1F}, C₁₋₄ alkoxy, C₁₋₄ alkyl, C₃₋₈ cycloalkyl, C₃₋₈ cycloalkyl-C₁₋₃ alkyl, C₁₋₄ alkanoyl, C₁₋₄ alkylsulphonyl, or C(O)NR^{1E}R^{1F}, NR^{1E}C(O)R^{1F}, NR^{1E}S(O)₂R^{1F} and S(O)₂NR^{1E}R^{1F};
R₄ is selected from hydrogen, cyano, halo, nitro, hydroxy or a group
-A²-B²-C²
wherein, A² is absent or a linker group of the formula —[CR^{2A}R^{2B}]_{q}— in which q is an integer selected from 1 or 2, 3 or 4, and R^{2A} and R^{2B} are each independently selected from hydrogen or C₁₋₂ alkyl;
B² is absent or —O—, —C(O)—, —C(O)O—, —OC(O)—, —CH(OR^{2C})—, —N(R^{2C})—, N(R^{2D})—C(O)—, —N(R^{2D})—C(O)O—, —C(O)—N(R^{2C})—, —N(R^{2D})C(O)N(R^{2C})—, —S—, —SO—, —SO₂—, —S(O)₂N(R^{2C})—, or —N(R^{2D})SO₂— wherein R^{2C} and R^{2D} are each independently selected from hydrogen or methyl; and
C² is hydrogen, C₁₋₆ alkyl, C₂₋₆ alkenyl, C₂₋₆ alkynyl, aryl, C₃₋₄ cycloalkyl, C₃₋₆ cycloalkenyl, heterocyclyl; or heteroaryl
and wherein C² is optionally further substituted by one or more substituent groups independently selected from oxo, halo, cyano, nitro, hydroxy, carboxy, NR^{2E}R^{2F}, C₁₋₄ alkoxy, C₃₋₈ cycloalkyl, C₁₋₄ alkyl, C₃₋₈ cycloalkyl-C₁₋₃ alkyl, C₁₋₄ alkylsulphonyl, C₁₋₄ alkanoyl, aryl, aryloxy, aryl-C₁₋₂ alkyl, heterocyclyl, heterocyclyloxy, heterocyclyl-C₁₋₂ alkyl, heteroaryl, heteroaryloxy, heteroaryl-C₁₋₂ alkyl, C(O)NR^{2E}R^{2F}, NR^{2E}C(O)R^{2F}, NR^{2E}S(O)₂R^{2F} and S(O)₂NR^{2E}R^{2F}; wherein R^{2E} and R^{2F} are each independently selected from hydrogen, C₁₋₄ alkyl or C₃₋₆ cycloalkyl or C₃₋₄ cycloalkyl C₁₋₂ alkyl; or R^{2E} and R^{2F} can be linked such that, together with the nitrogen atom to which they are attached, they form a 4-7 membered heterocyclic ring;
and wherein any alkyl, aryl, heterocyclyl or heteroaryl group present in a substituent group on C² is optionally further substituted by halo, cyano, nitro, hydroxy, caboxy, NR^{2G}R^{2H}, C₁₋₂ alkoxy, or C₁₋₂ alkyl; wherein R^{2G} and R^{2H} are selected from hydrogen or C₁₋₂ alkyl;
or R^{2C} and C² can be linked such that, together with the nitrogen atom to which they are attached, they form a 4-7 membered heterocyclic ring, which is optionally substituted by oxo, halo, cyano, nitro, hydroxy, carboxy, NR^{2E}R^{2F}, C₁₋₄ alkoxy, C₁₋₄ alkyl, C₃₋₈ cycloalkyl, C₃₋₈ cycloalkyl-C₁₋₃ alkyl, C₁₋₄ alkanoyl, C₁₋₄ alkylsulphonyl, or C(O)NR^{2E}R^{2F}, NR^{2E}C(O)R^{2F}, NR^{2E}S(O)₂R^{2F} and S(O)₂NR^{2E}R^{2F};
R₅ is selected from hydrogen, halo, cyano, hydroxy, nitro, or a group
-A³-B³-C³
wherein, A³ is absent or a linker group of the formula —[CR^{3A}R^{3B}]ᵣ—in which r is an integer selected from 1 or 2, 3 or 4, and R^{3A} and R^{3B} are each independently selected from hydrogen or C₁₋₂ alkyl;
B³ is absent or —O—, —C(O)—, —C(O)O—, —OC(O)—, —CH(OR^{3C})—, —N(R^{3C})—, N(R^{3D})—C(O)—, —N(R^{3D})—C(O)O—, —C(O)—N(R^{3C})—, —N(R^{3D})C(O)N(R^{3C})—, —S—, —SO—, —SO₂—, —S(O)₂N(R^{3C})—, or —N(R^{3D})SO₂— wherein R^{3C} and R^{3D} are each independently selected from hydrogen or methyl; and
C³ is hydrogen, C₁₋₄ alkyl, C₂₋₆ alkenyl, C₂₋₆ alkynyl, aryl, C₃₋₄ cycloalkyl, C₃₋₄ cycloalkenyl, heteroaryl or heterocyclyl;
and wherein C³ is optionally further substituted by one or more substituent groups independently selected from oxo, halo, cyano, nitro, hydroxy, carboxy, NR^{3E}R^{3F}, C₁₋₄ alkoxy, C₃₋₈ alkyl, C₃₋₈ cycloalkyl, C₃₋₈ cycloalkyl-C₁₋₃ alkyl, C₁₋₄ alkanoyl, C₁₋₄ alkylsulphonyl, aryl, aryloxy, aryl-C₁₋₂ alkyl, heterocyclyl, heterocyclyloxy, heterocyclyl-C₁₋₂ alkyl, heteroaryl, heteroaryloxy, heteroaryl-C₁₋₂ alkyl, C(O)NR^{3E}R^{3F}, NR^{3E}C(O)R^{3F}, NR^{3E}S(O)₂R^{3F} and S(O)₂NR^{3E}R^{3F}; wherein R^{3E} and R^{3F} are each independently selected from hydrogen, C₁₋₄ alkyl or C₃₋₆ cycloalkyl or C₃₋₆ cycloalkyl C₁₋₂ alkyl; or R^{3E} and R^{3F} can be linked such that, together with the nitrogen atom to which they are attached, they form a 4-7 membered heterocyclic ring;
and wherein any alkyl, aryl, heterocyclyl or heteroaryl group present in a substituent group on C³ is optionally further substituted by halo, nitro, cyano, hydroxy, caboxy, NR^{3G}R^{3H}, C₁₋₂ alkoxy, or C₁₋₂ alkyl; wherein R^{3G} and R^{3H} are selected from hydrogen or C₁₋₂ alkyl;
or R^{3C} and C³ can be linked such that, together with the nitrogen atom to which they are attached, they form a 4-7 membered heterocyclic ring, which is optionally substituted by oxo, halo, cyano, hydroxy, nitro, carboxy, NR^{3E}R^{3F}, C₁₋₄ alkoxy, C₁₋₄ alkyl, C₃₋₈ cycloalkyl, C₃₋₈ cycloalkyl-C₁₋₃ alkyl, C₁₋₄ alkanoyl, C₁₋₄ alkylsulphonyl, or C(O)NR^{3E}R^{3F}, NR^{3E}C(O)R^{3F}, NR^{3E}S(O)₂R^{3F} and S(O)₂NR^{3E}R^{3F};
R₆ is selected from hydrogen, halo, cyano, nitro, hydroxy or a group
-A⁴-B⁴-C⁴
wherein, A⁴ is absent or a linker group of the formula —[CR^{4A}R^{4B}]ₛ— in which is an integer selected from 1 or 2, 3 or 4, and R^{4A} and R^{4B} are each independently selected from hydrogen or C₁₋₂ alkyl;
B⁴ is absent or —O—, —C(O)—, —C(O)O—, —OC(O)—, —CH(OR^{4C})—, —N(R^{4C})—, N(R^{4D})—C(O)—, —N(R^{4D})—C(O)O—, —C(O)—N(R^{4C})—, —N(R^{4D})C(O)N(R^{4C})—, —S—, —SO—, —SO₂—, —S(O)₂N(R^{4C})—, or —N(R^{4D})SO₂— wherein R^{4C} and R^{4D} are each independently selected from hydrogen or methyl; and
C⁴ is hydrogen, C₁₋₆ alkyl, C₂₋₆ alkenyl, C₂₋₆ alkynyl, aryl, C₃₋₆ cycloalkyl, C₃₋₆ cycloalkenyl, heterocyclyl, or heteroaryl,
and wherein C⁴ is optionally further substituted by one or more substituent groups independently selected from oxo, halo, cyano, nitro, hydroxy, carboxy, NR^{4E}R^{4F}, C₁₋₄ alkoxy, C₃₋₈ alkyl, C₃₋₈ cycloalkyl, C₃₋₈ cycloalkyl-C₁₋₃ alkyl, C₁₋₄ alkanoyl, C₁₋₄ alkylsulphonyl, aryl, aryloxy, aryl-C₁₋₂ alkyl, heterocyclyl, heterocyclyloxy, heterocyclyl-C₁₋₂ alkyl, heteroaryl, heteroaryloxy, heteroaryl-C₁₋₂ alkyl, C(O)NR^{4E}R^{4F}, NR^{4E}C(O)R^{4F}, NR^{4E}S(O)₂R^{4F} and S(O)₂NR^{4E}R^{4F}; wherein R^{4E} and R^{4F} are each independently selected from hydrogen, C₁₋₄ alkyl or C₃₋₆ cycloalkyl or C₃₋₆ cycloalkyl C₁₋₂ alkyl; or R^{4E} and R^{4F} can be linked such that, together with the nitrogen atom to which they are attached, they form a 4-7 membered heterocyclic ring;
and wherein any alkyl, aryl, heterocyclyl or heteroaryl group present in a substituent group on C⁴ is optionally further substituted by halo, cyano, nitro, hydroxy, caboxy, NR^{4G}R^{4H}, C₁₋₂ alkoxy, or C₁₋₂ alkyl; wherein R^{4G} and R^{4H} are selected from hydrogen or C₁₋₂ alkyl;
or R^{4C} and C⁴ can be linked such that, together with the nitrogen atom to which they are attached, they form a 4-7 membered heterocyclic ring, which is optionally substituted by oxo, halo, cyano, nitro, hydroxy, carboxy, NR^{4E}R^{4F}, C₁₋₄alkoxy, C₁₋₄ alkyl, C₃₋₈cycloalkyl, C₃₋₈cycloalkyl-C₁₋₃alkyl, C₁₋₄ alkanoyl, C₁₋₄ alkylsulphonyl, or C(O)NR^{4E}R^{4F}, NR^{4E}C(O)R^{4F}, NR^{4E}S(O)₂R^{4F} and S(O)₂NR^{4E}R^{4F};
R₇ is selected from hydrogen, halo, nitro, cyano, hydroxy or a group
-A⁵-B⁵-C⁵
wherein, A⁵ is absent or a linker group of the formula —[CR^{5A}R^{5B}]ₜ— in which ‘t’ is an integer selected from 1 or 2, 3 or 4, and R^{5A} and R^{5B} are each independently selected from hydrogen or C₁₋₂ alkyl;
B⁵ is absent or —O—, —C(O)—, —C(O)O—, —OC(O)—, —CH(OR^{5C})—, —N(R^{5C})—, N(R^{5D})—C(O)—, —N(R^{5D})—C(O)O—, —C(O)—N(R^{5C})—, —N(R^{5D})C(O)N(R^{5C})—, —S—, —SO—, —SO₂—, —S(O)₂N(R^{5C})—, or —N(R^{5D})SO₂— wherein R^{5C} and R^{5D} are each independently selected from hydrogen or methyl; and
C⁵ is hydrogen, C₁₋₄ alkyl, C₂₋₆ alkenyl, C₂₋₆ alkynyl, aryl, C₃₋₆ cycloalkyl, C₃₋₆ cycloalkenyl, heteroaryl or heterocyclyl;
and wherein C⁵ is optionally further substituted by one or more substituent groups independently selected from oxo, cyano, halo, nitro, carboxy, hydroxy, NR^{5E}R^{5F}, C₁₋₄ alkoxy, C₁₋₄ alkyl, C₃₋₈ cycloalkyl, C₃₋₈ cycloalkyl-C₁₋₃ alkyl, C₁₋₄ alkanoyl, C₁₋₄ alkylsulphonyl, aryl, aryloxy, aryl-C₁₋₂ alkyl, heterocyclyl, heterocyclyloxy, heterocyclyl-C₁₋₂alkyl, heteroaryl, heteroaryloxy, heteroaryl-C₁₋₂alkyl, C(O)NR^{5E}R^{5F}, NR^{5E}C(O)R^{5F}, NR^{5E}S(O)₂R^{5F} and S(O)₂NR^{5E}R^{5F}; wherein R^{5E} and R^{5F} are each independently selected from hydrogen, C₁₋₄ alkyl or C₃₋₆ cycloalkyl or C₃₋₄ cycloalkyl C₁₋₂ alkyl; or R^{5E} and R^{5F} can be linked such that, together with the nitrogen atom to which they are attached, they form a 4-7 membered heterocyclic ring;
and wherein any alkyl, aryl, heterocyclyl or heteroaryl group present in a substituent group on C⁵ is optionally further substituted by halo, cyano, nitro, hydroxy, caboxy, NR^{5G}R^{5H}, C₁₋₂ alkoxy, or C₁₋₂ alkyl;
wherein R^{5G} and R^{5H} are selected from hydrogen or C₁₋₂ alkyl;
or R^{5C} and C⁵ can be linked such that, together with the nitrogen atom to which they are attached, they form a 4-7 membered heterocyclic ring, which is optionally substituted by oxo, halo, cyano, nitro, hydroxy, carboxy, NR^{5E}R^{5F}, C₁₋₄ alkoxy, C₁₋₄ alkyl, C₃₋₈ cycloalkyl, C₃₋₈ cycloalkyl-C₁₋₃ alkyl, C₁₋₄ alkanoyl, C₁₋₄ alkylsulphonyl, or C(O)NR^{5E}R^{5F}, NR^{5E}C(O)R^{5F}, NR^{5E}S(O)₂R^{5F} and S(O)₂NR^{5E}R^{5F};
R₈ and R₉, are selected from group, which may be same or different at each occurrence, are independently selected from hydrogen, hydroxy, halogen, cyano, substituted or unsubstituted haloalkyl, substituted or unsubstituted alkyl and substituted or unsubstituted cycloalkyl; or R₈ and R₉ together with the carbon atom to which they are attached, may form a substituted or unsubstituted 3 to 7 membered saturated carbocyclic ring;
R₁₀ and R₁₁, which may be same or different at each occurrence, are independently selected from hydrogen, cyano, halogen, hydroxy, substituted or unsubstituted haloalkyl, substituted or unsubstituted alkyl and substituted or unsubstituted cycloalkyl; or R₁₀ and R₁₁ together with the carbon atom to which they are attached, may form a substituted or unsubstituted 3 to 7 membered saturated carbocyclic ring.

2. The compound as claimed in claim 1, wherein compound of formula I is selected from the group consisting of:
1. Methyl 3-(3,5-dichlorophenyl)-4,5-dihydro-1H-benzo[g]indole-2-carboxylate;
2. Methyl 3-(3,5-dichlorophenyl)-4,5-dihydro-1H-benzo[g]indole-2-carboxylic acid;
3. Methyl 3-(5-fluoro-2-methoxyphenyl)-4,5-dihydro-1H-benzo[g]indole-2-carboxylate;
4. Methyl 3-(2-methoxyphenyl)-4,5-dihydro-1H-benzo[g]indole-2-carboxylate;
5. Methyl 3-(4-methoxyphenyl)-4,5-dihydro-1H-benzo[g]indole-2-carboxylate;
6. Methyl 3-(3,4-difluorophenyl)-4,5-dihydro-1H-benzo[g]indole-2-carboxylate;
7. Methyl 3-(5-chloro-2-methoxyphenyl)-4,5-dihydro-1H-benzo[g]indole-2-carboxylate;
8. Methyl 3-(4-(tert-butyl)phenyl)-4,5-dihydro-1H-benzo[g]indole-2-carboxylate;
9. Methyl 3-(4-fluorophenyl)-4,5-dihydro-1H-benzo[g]indole-2-carboxylate;
10. Methyl 3-phenyl-4,5-dihydro-1H-benzo[g]indole-2-carboxylate;
11. Methyl 3-(2-fluorophenyl)-4,5-dihydro-1H-benzo[g]indole-2-carboxylate;
12. Methyl 3-(3-fluorophenyl)-4,5-dihydro-1H-benzo[g]indole-2-carboxylate;
13. Methyl 3-(4-chlorophenyl)-4,5-dihydro-1H-benzo[g]indole-2-carboxylate;
14. Methyl 3-(2-chlorophenyl)-4,5-dihydro-1H-benzo[g]indole-2-carboxylate;
15. Methyl 3-(3-chlorophenyl)-4,5-dihydro-1H-benzo[g]indole-2-carboxylate;
16. Methyl 3-(2,4-dichlorophenyl)-4,5-dihydro-1H-benzo[g]indole-2-carboxylate;
17. Methyl 3-(2,3-difluorophenyl)-4,5-dihydro-1H-benzo[g]indole-2-carboxylate;
18. Methyl 3-(3,5-dimethylphenyl)-4,5-dihydro-1H-benzo[g]indole-2-carboxylate;
19. Methyl 3-(2-fluoro-4-methylphenyl)-4,5-dihydro-1H-benzo[g]indole-2-carboxylate;
20. Methyl 3-(2,3-dichlorophenyl)-4,5-dihydro-1H-benzo[g]indole-2-carboxylate;
21. Methyl 3-(pyridin-4-yl)-4,5-dihydro-1H-benzo[g]indole-2-carboxylate;
22. Methyl 3-(3,5-difluorophenyl)-4,5-dihydro-1H-benzo[g]indole-2-carboxylate;
23. Methyl 3-(pyrimidin-5-yl)-4,5-dihydro-1H-benzo[g]indole-2-carboxylate;
24. Methyl 3-(5-fluoropyridin-3-yl)-4,5-dihydro-1H-benzo[g]indole-2-carboxylate;
25. Methyl 3-(5-chloropyridin-3-yl)-4,5-dihydro-1H-benzo[g]indole-2-carboxylate;
26. Methyl 3-(4-fluoro-3-methoxyphenyl)-4,5-dihydro-1H-benzo[g]indole-2-carboxylate;
27. Methyl 3-(3-chloro-5-methylphenyl)-4,5-dihydro-1H-benzo[g]indole-2-carboxylate;
28. Methyl 3-(3-chloro-5-fluorophenyl)-4,5-dihydro-1H-benzo[g]indole-2-carboxylate;
29. Methyl 3-(3-chloro-5-methoxyphenyl)-4,5-dihydro-1H-benzo[g]indole-2-carboxylate;
30. Methyl 3-(3,4,5-trichlorophenyl)-4,5-dihydro-1H-benzo[g]indole-2-carboxylate;
31. Methyl 3-(3,5-dimethoxyphenyl)-4,5-dihydro-1H-benzo[g]indole-2-carboxylate;
32. Methyl 3-(3-fluoro-5-methoxyphenyl)-4,5-dihydro-1H-benzo[g]indole-2-carboxylate;
33. Methyl 3-(2,3-dichlorophenyl)-4,5-dihydro-1H-benzo[g]indole-2-carboxylate;
34. 3-(5-Fluoro-2-methoxyphenyl)-4,5-dihydro-1H-benzo[g]indole-2-carboxylic acid;
35. 3-(2-methoxyphenyl)-4,5-dihydro-1H-benzo[g]indole-2-carboxylic acid;
36. 3-(4-Methoxyphenyl)-4,5-dihydro-1H-benzo[g]indole-2-carboxylic acid;
37. 3-(3,4-Difluorophenyl)-4,5-dihydro-1H-benzo[g]indole-2-carboxylic acid;
38. 3-(5-Chloro-2-methoxyphenyl)-4,5-dihydro-1H-benzo[g]indole-2-carboxylic acid;
39. 3-(4-(tert-Butyl)phenyl)-4,5-dihydro-1H-benzo[g]indole-2-carboxylic acid;
40. 3-(4-Fluorophenyl)-4,5-dihydro-1H-benzo[g]indole-2-carboxylic acid;
41. 3-(2-Fluorophenyl)-4,5-dihydro-1H-benzo[g]indole-2-carboxylic acid;
42. 3-(3-Fluorophenyl)-4,5-dihydro-1H-benzo[g]indole-2-carboxylic acid;
43. 3-(4-chlorophenyl)-4,5-dihydro-1H-benzo[g]indole-2-carboxylic acid;
44. 3-(2-chlorophenyl)-4,5-dihydro-1H-benzo[g]indole-2-carboxylic acid;
45. 3-(3-chlorophenyl)-4,5-dihydro-1H-benzo[g]indole-2-carboxylic acid;
46. 3-(2,4-dichlorophenyl)-4,5-dihydro-1H-benzo[g]indole-2-carboxylic acid;
47. 3-(2,3-difluorophenyl)-4,5-dihydro-1H-benzo[g]indole-2-carboxylic acid;
48. 3-(3,5-dimethylphenyl)-4,5-dihydro-1H-benzo[g]indole-2-carboxylic acid;
49. 3-(2-fluoro-4-methylphenyl)-4,5-dihydro-1H-benzo[g]indole-2-carboxylic acid;
50. 3-(pyridin-4-yl)-4,5-dihydro-1H-benzo[g]indole-2-carboxylic acid;
51. 3-(3,5-difluorophenyl)-4,5-dihydro-1H-benzo[g]indole-2-carboxylic acid;
52. 3-(5-fluoropyridin-3-yl)-4,5-dihydro-1H-benzo[g]indole-2-carboxylic acid;
53. 3-(5-chloropyridin-3-yl)-4,5-dihydro-1H-benzo[g]indole-2-carboxylic acid;
54. 3-(2,3-dichlorophenyl)-4,5-dihydro-1H-benzo[g]indole-2-carboxylic acid;
55. 3-(pyrimidin-5-yl)-4,5-dihydro-1H-benzo[g]indole-2-carboxylic acid;
56. 3-(4-fluoro-2-methoxyphenyl)-4,5-dihydro-1H-benzo[g]indole-2-carboxylic acid;
57. 3-(3-chloro-5-methylphenyl)-4,5-dihydro-1H-benzo[g]indole-2-carboxylic acid;
58. 3-(3-chloro-5-fluorophenyl)-4,5-dihydro-1H-benzo[g]indole-2-carboxylic acid;
59. 3-(3-chloro-5-methoxyphenyl)-4,5-dihydro-1H-benzo[g]indole-2-carboxylic acid;
60. 3-(3,4,5-trichlorophenyl)-4,5-dihydro-1H-benzo[g]indole-2-carboxylic acid;
61. 3-(3,5-dimethoxyphenyl)-4,5-dihydro-1H-benzo[g]indole-2-carboxylic acid;
62. 3-(3-fluoro-5-methoxyphenyl)-4,5-dihydro-1H-benzo[g]indole-2-carboxylic acid;
63. 3-phenyl-4,5-dihydro-1H-benzo[g]indole-2-carboxylic acid;
64. 3-(3-chloro-5-fluorophenyl)-5-methyl-4,5-dihydro-1H-benzo[g]indole-2-carboxylic acid;
65. 7-bromo-3-(3,5-dichlorophenyl)-4,5-dihydro-1H-benzo[g]indole-2-carboxylic acid.

3. A process for the preparation of compound of formula I as claimed in claim 1 comprising the steps of:
i. reducing tetralone of formula (1) using reducing agents such as sodium borohydride to give compound of formula (2); 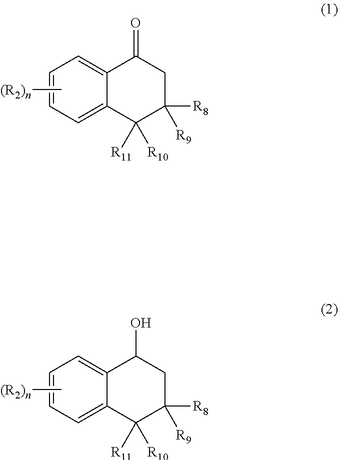
ii. formylation of compound of formula (2) as obtained in step (i) using in-situ prepared Vilsmeier Haack reagent/adduct to give compound of formula (3); 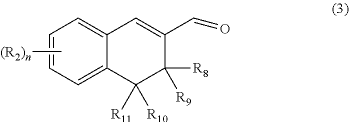
iii. treating aldehyde of formula (3) as obtained in step (ii) with azido ester compound of formula (4) in the presence of a base such as sodium ethoxide, sodium methoxide etc. to give compound of formula (5); 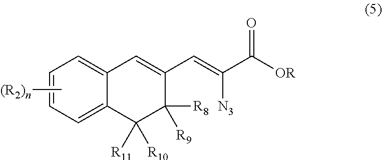
iv. cyclization of compound of formula (5) to get the compound of the formula (6) in the presence of suitable Lewis acid or in acidic condition; 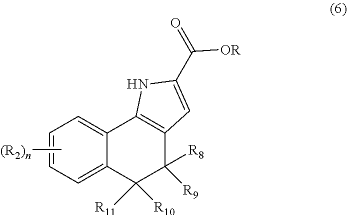
v. halogenating the compound of formula (6) using halogenated reagents such as NBS, NIS, etc. to give the halogenated compound of the formula (7); 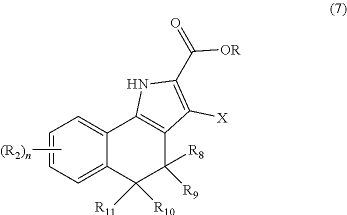
vi. coupling reaction of the halo compound of formula (7) with aryl/heteroaryl boronic acids or aryl/heteroaryl boronic esters to give compound of Formula (Ia); 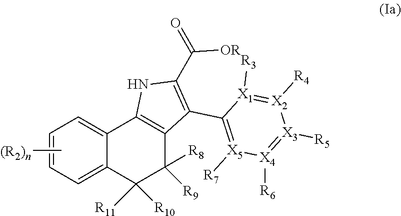
vii. converting compound of Formula (Ia) by hydrolysis of corresponding ester using base such as LiOH, K₂CO₃, NaOH to compound of Formula (I); 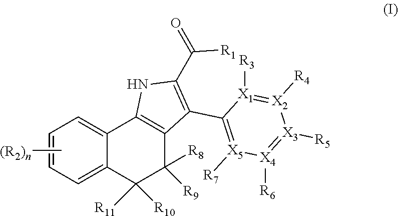
viii. In acid of formula (I) can be converted —COOH into corresponding amide, ester etc., wherein R1 is same as defined above.

4. A compound of formula I according to claim 1 in free form or in salt form or in pharmaceutically acceptable salt form.

5. A pharmaceutical composition comprising at least one compound of Formula (I) optionally along with pharmaceutically acceptable excipient.

6. The pharmaceutical composition as claimed in claim 1, wherein said composition further comprises an effective amount of a beta-lactam antibiotic.

7. The pharmaceutical composition as claimed in claim 1, wherein said composition is for use as a beta-lactamase inhibitor or as a drug.

8. Use of a compound of formula (I), or a pharmaceutically acceptable salt thereof, for inhibiting beta-lactamase activity, in the manufacture of a medicament for inhibiting beta-lactamase activity, in combination with a beta-lactam antibiotic for treating a bacterial infection, or in combination with a beta-lactam antibiotic in the manufacture of a medicament for treating a bacterial infection.
